(19) United States
(12) Patent Application Publication
(10) Pub. No.: US 2020/0255867 A1
(43) Pub. Date: Aug. 13, 2020

(54) DOUBLE SELECTION HDR CRISPR-BASED EDITING

(71) Applicant: THE BROAD INSTITUTE, INC., Cambridge, MA (US)

(72) Inventors: Melina CLAUSSNITZER, Cambridge, MA (US); Sarah GOGGIN, Cambridge, MA (US); Alham SAADAT, Cambridge, MA (US)

(21) Appl. No.: 16/643,251

(22) PCT Filed: Aug. 30, 2018
(86) PCT No.: PCT/US2018/048943

§ 371 Date: Feb. 28, 2020

Related U.S. Application Data
(60) Provisional application No. 62/552,333, filed on Aug. 30, 2017.

(51) Int. Cl. C12N 15/90; C12N 15/86; C12N 15/11; C12Q 1/6883; C12Q 1/6888

(52) U.S. Cl. CPC C12N 15/907; C12N 15/86; C12N 15/11; C12N 2310/20; C12Q 1/6888; C12N 2740/15043; C12Q 1/6883

(57) ABSTRACT

The invention provides homology directed repair (HDR) constructs for variant screening in cells comprising: a left and right homology arm, with either the left or right homology arm encoding a genomic edit to be incorporated at a target locus; and an excisable double selection cassette located within the left and right homology arms, the excisable double selection cassette comprising; a first selection marker; and a second selection marker; and wherein the first selection marker and the second selection marker are located between a first and second excision site. Also provided are homology directed repair (HDR) vectors comprising a construct as described herein, and methods for using such vectors.

### CROSS REFERENCE TO RELATED APPLICATION

This application claims the benefit of U.S. Provisional Application No. 62/552,333, filed Aug. 30, 2017. The entire contents of the above-identified application are hereby fully incorporated herein by reference.

### STATEMENT REGARDING FEDERALLY SPONSORED RESEARCH

This invention was made with government support under grant number HG008155 granted by the National Institutes of Health. The government has certain rights in the invention.

### TECHNICAL FIELD

The subject matter disclosed herein is generally directed to constructs, systems, and methods for screening for genomic variants of diverse cellular and organismal phenotypes.

### BACKGROUND

Advances in genome analysis techniques have significantly accelerated the ability to catalog and map genetic factors associated with a diverse range of biological functions and diseases. Precise genome targeting technologies are needed to enable systematic reverse engineering of causal genetic variations by allowing selective perturbation of individual genetic elements, as well as to advance synthetic biology, biotechnological, and medical applications. Although genome-editing techniques such as designer zinc fingers, transcription activator-like effectors (TALEs), or homing meganucleases are available for producing targeted genome perturbations, there remains a need for new genome engineering technologies that employ novel strategies and molecular mechanisms and are affordable, easy to set up, scalable, and amenable to targeting multiple positions within the eukaryotic genome. This would provide a major resource for new applications in genome engineering and biotechnology.

The CRISPR-CRISPR associated (Cas) systems of bacterial and archaeal adaptive immunity are some such systems that show extreme diversity of protein composition and genomic loci architecture. The CRISPR-Cas system locus has more than 50 gene families and there are no strictly universal genes, indicating fast evolution and extreme diversity of locus architecture. A novel genome editing method and related constructs and vectors utilizing the CRISPR-Cas system is provided that enables editing at the single nucleotide level.

### SUMMARY

In one aspect, the invention provides a homology directed repair (HDR) construct for variant screening in cells comprising: a left and right homology arm, with either the left or right homology arm encoding a genomic edit to be incorporated at a target locus; and an excisable double selection cassette located within the left and right homology arms, the excisable double selection cassette comprising; a first selection marker; and a second selection marker; and a fluorescent marker; and wherein the first selection marker and the second selection marker and the fluorescent marker are located between a first and second excision site. In one embodiment, the first and second selection markers are a positive selection marker and a negative selection marker, respectively. In another embodiment, the positive selection marker is a drug resistance gene. In certain example embodiments, the positive selection marker is a puromycin resistance gene, a zeocin resistance gene, a blasticidin resistance gene, a geneticin (G-418) resistance gene, or a hygromycin B resistance gene. In another embodiment, an HDR construct may further comprise a fluorescent marker for isolation or quantification of positive cell pools. In certain example embodiments, the selectable marker is suitable for FACS isolation. In certain example embodiments, the fluorescent marker comprises BFP, Cyan-Cerulean, GFP2, YPet, RFP, Far Red-mKate2. In another embodiment, the left and right homology arms are each from about 700 bp to about 1000 bp. In another embodiment, the second selection maker is a drug sensitivity gene, such as a thymidine kinase gene. In still further embodiments, the first and second excision sites are transposase recognition sites.

In another aspect, the invention provides a homology directed repair (HDR) vector comprising a construct as described herein. In one embodiment, the backbone of the vector enables uniform, one-step assembly for incorporating homology arms. In another embodiment, the vector is a transfection delivery vector. In another embodiment, the vector is a viral delivery vector. In a further embodiment, the viral delivery vector is a lentivirus vector.

In another aspect, the invention provides a variant screening system for screening cells comprising: a gene editing system; a HDR vector as described herein; and an excision protein or a polynucleotide encoding an excision protein, wherein the excision protein removes the excisable double selection cassette. In one embodiment, the gene editing system comprises a CRISPR system comprising a CRISPR effector protein and/or a polynucleotide encoding the CRISPR effector protein, and a guide RNA (gRNA) comprising a guide sequence and/or a polynucleotide encoding the gRNA, wherein the gRNA is capable of forming a complex with the CRISPR effector protein and binding a target sequence adjacent to a variant locus to be edited. In another embodiment, such a system comprises two or more delivery vectors, each delivery vector comprising a guide RNA targeted to a different variant locus. In another embodiment, such a system comprises two or more HDR vectors wherein each HDR vector encodes a different nucleotide edit at each variant locus, each with different positive selection marker and fluorescent marker pairs. In another embodiment, the excision protein is a transposase, such as an excision transposase, or a hyperactive transposase, or the transposase comprises a mutation that alters its function. In a specific embodiment, the transposase comprises a PiggyBac transposase.

In another aspect, the invention provides a method for screening variant loci in cells comprising: delivering one or more HDR constructs as described herein and/or one or more HDR delivery vectors as described herein to: (i) a population of cells expressing a gene editing system configured to modify cellular DNA at one or more target loci; or (ii) a population of cells to which a gene editing system configured to modify cellular DNA at one or more target loci is co-delivered with the HDR construct or the HDR delivery vector; selecting edited cells that incorporate the excisable double selection cassette of the HDR construct based on the first selection marker; selecting a final cell population based on the second selection marker; and delivering an excision protein, or a polynucleotide encoding the excision protein, to the edited cells, wherein the excision protein removes the excisable double selection cassette, to arrive at a final edited cell population. In one embodiment, the gene editing system comprises a CRISPR system. In another embodiment, the method further comprises a quality control or genotyping step after the first selecting step, the second selecting step, or both. In another embodiment, the QC/genotyping step can be used to quantify the percentage of edited cells in pre- or post-selection cell populations. In a further embodiment, the QC/genotyping step comprises fluorescence-based cell counting or FACS. In a still further embodiment, the QC/genotyping step comprises amplicon sequencing. In a still further embodiment, the method further comprises determining changes in expression of one or more biomarkers in the final edited cell population and/or changes in one or more cellular phenotypes of the final edited cell population. In another embodiment, the one or more changes in cellular phenotype include changes in morphology, motility, cell death, cell-cell contact or a combination thereof. In another embodiment, the one or more biomarkers are indicative of a presence or absence of a disease state or identify a cell type or cell lineage.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

FIG. 1—Shows a map of the pFUGW-PB-2XSelect vector. The protocol that was used for double selection base editing is shown in FIG. 2 and described in the Examples.

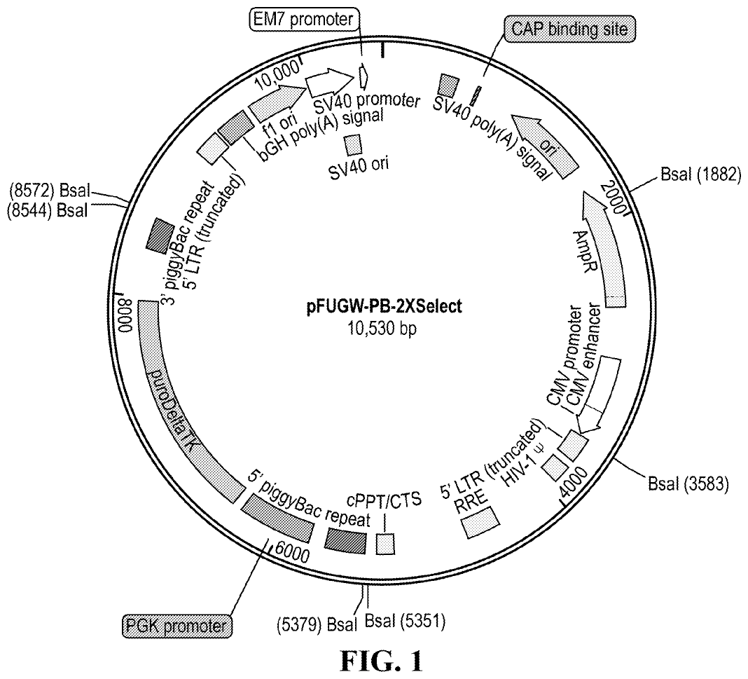

FIG. 2—Shows a flow chart for double selection base editing.

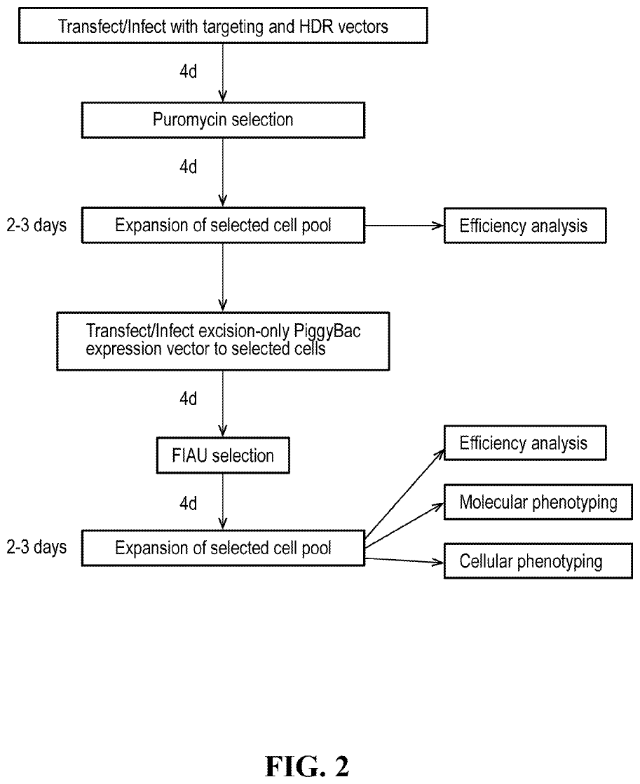

FIG. 3—Shows a sample viral vector for introduction into a construct of the invention.

FIG. 4—Shows a map of the gene sequence for the mutated hyperactive excision-only PB transposase.

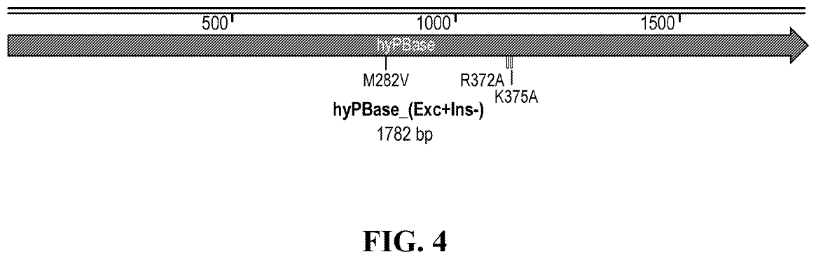

FIG. 5—Shows a map of the final lentiviral construct containing the mutated hyperactive excision-only PiggyBac transposase.

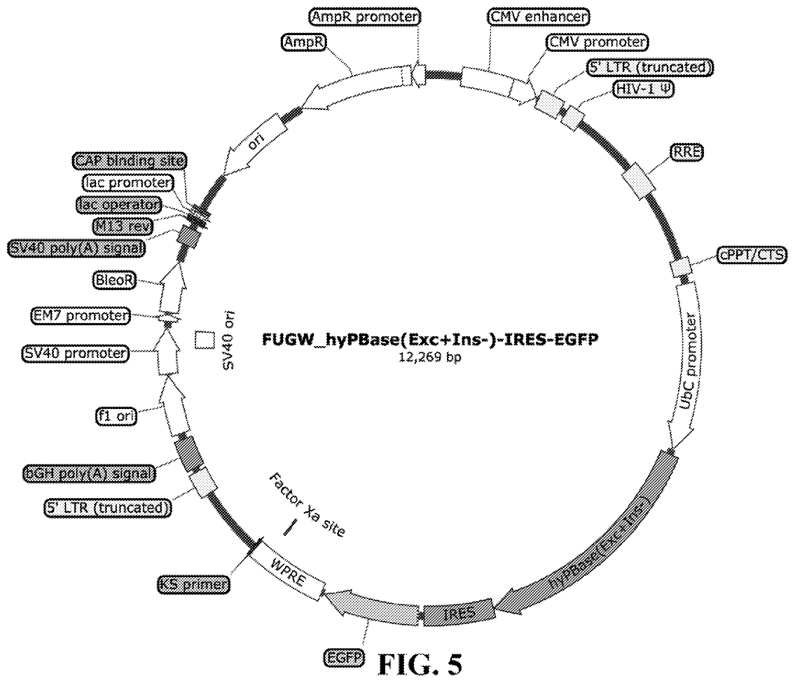

FIG. 6—Shows predicted causal variants identified in primary human PBMCs.

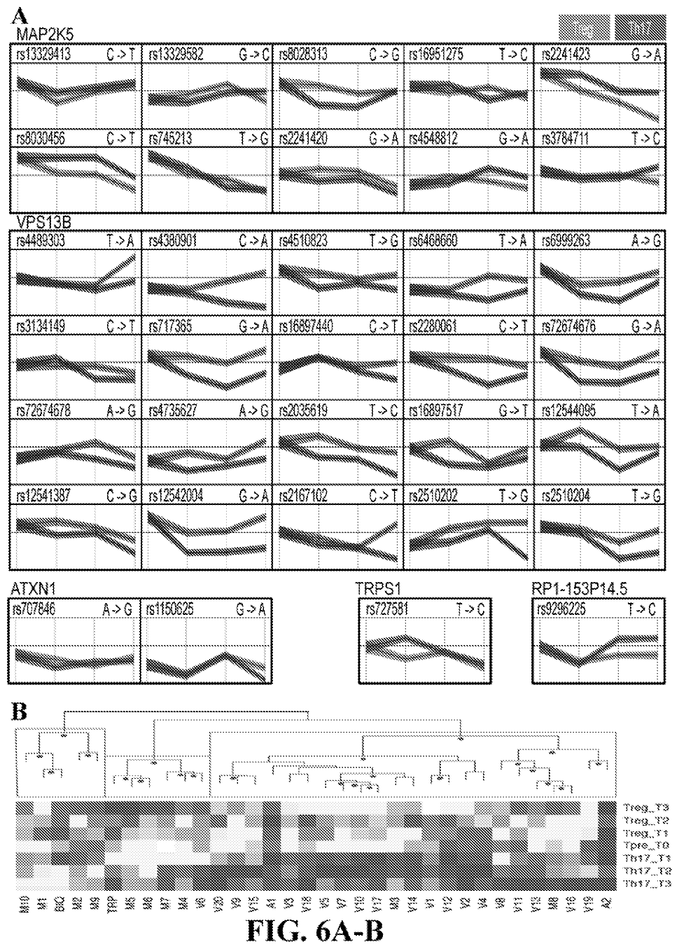

FIG. 7—Shows an overview of CRISPR-SAVE process of the present invention and data generated in accordance with certain example embodiments.

FIG. 8—Shows depictions of (a) a target variant, (b) homology-directed repair, (c) excision only transposase, and (d) scarless edit.

FIG. 9—Shows results of analyses as described in the Examples and in accordance with certain example embodiments.

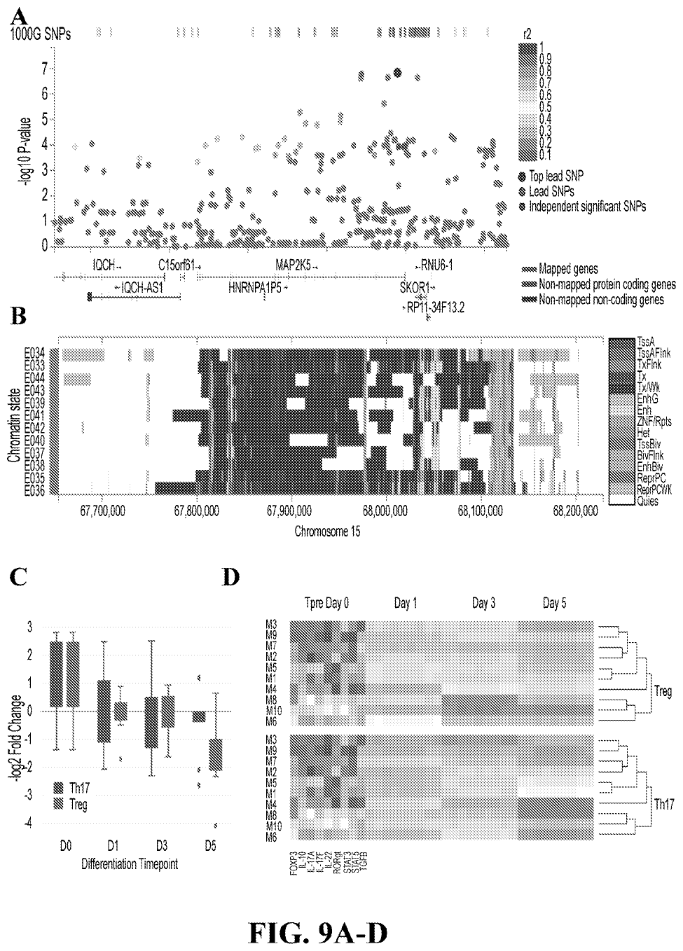

FIG. 10—Shows depictions of a target variant, a CRISPR break, homology-directed repair, insertion positive selection, excision negative selection, and scarless edit.

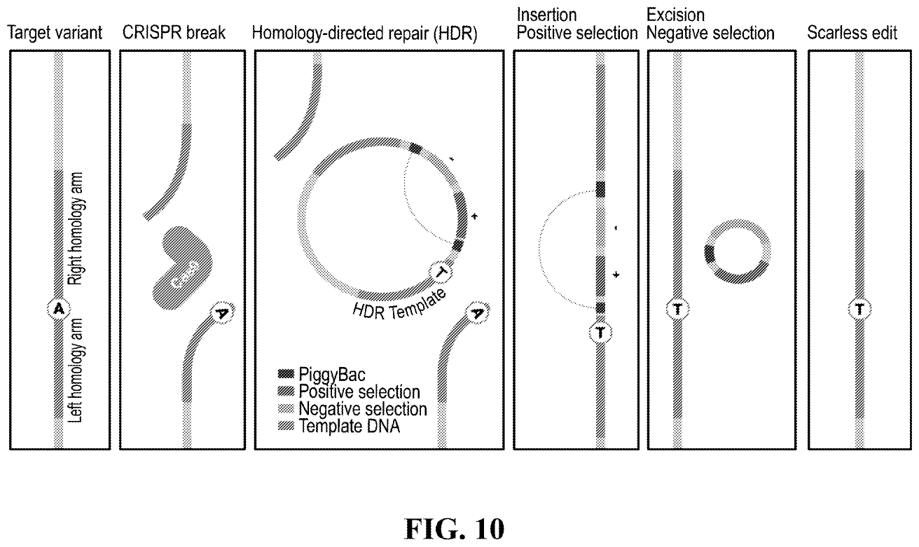

FIG. 11—Shows a map of the pMiniT-PuroTk-EGFP vector.

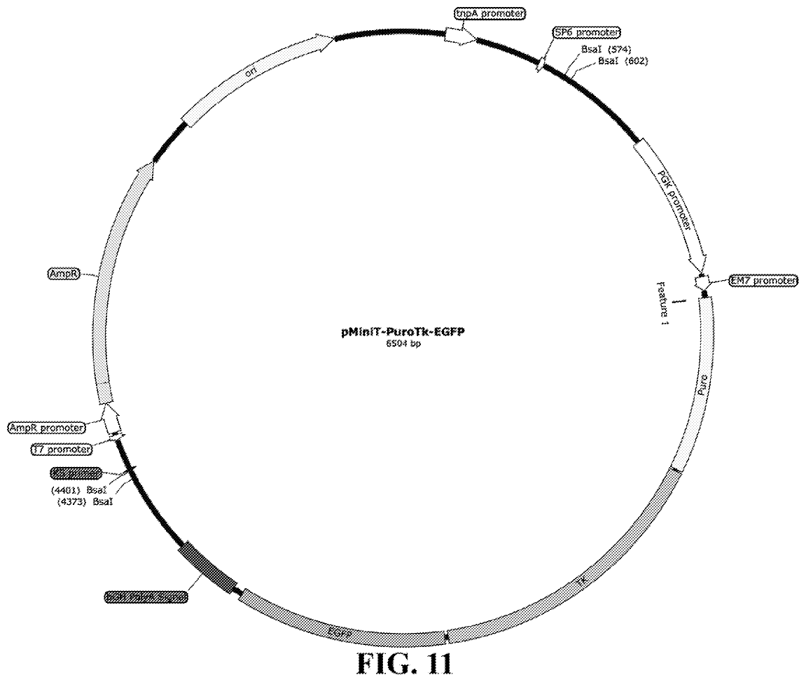

FIG. 12—Shows a map of the pFUGW-PuroTk-EGFP vector.

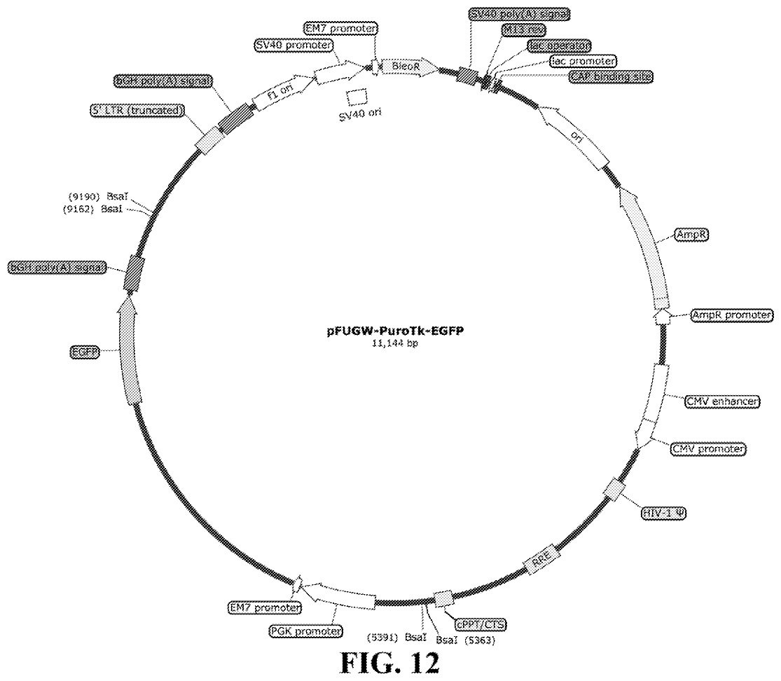

FIGS. 13A, 13B—Shows a sample viral vector for introduction into a construct of the invention.

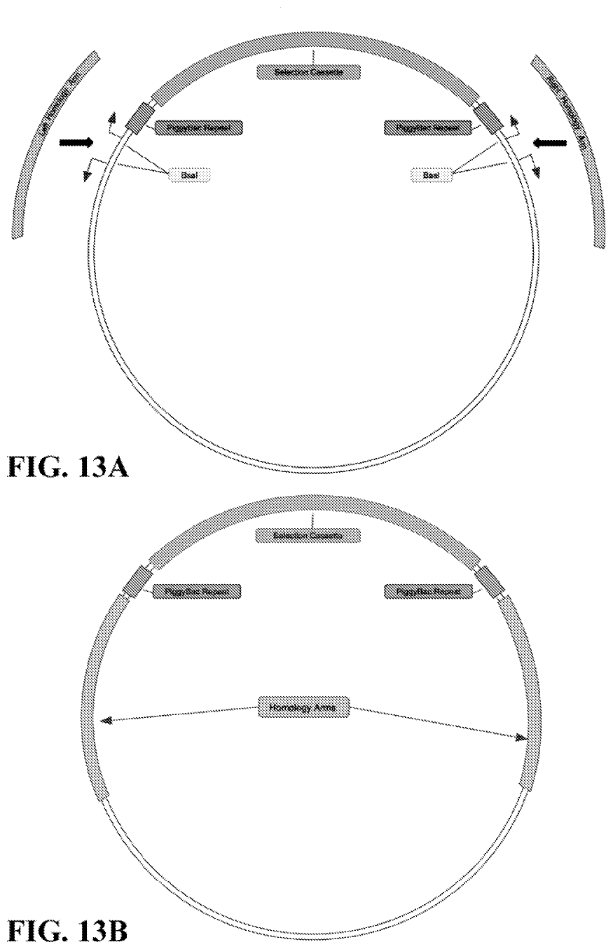

FIG. 14 shows a map of the construct expressing the hyperactive excision-only PB transposase (pCMV-hyPBase).

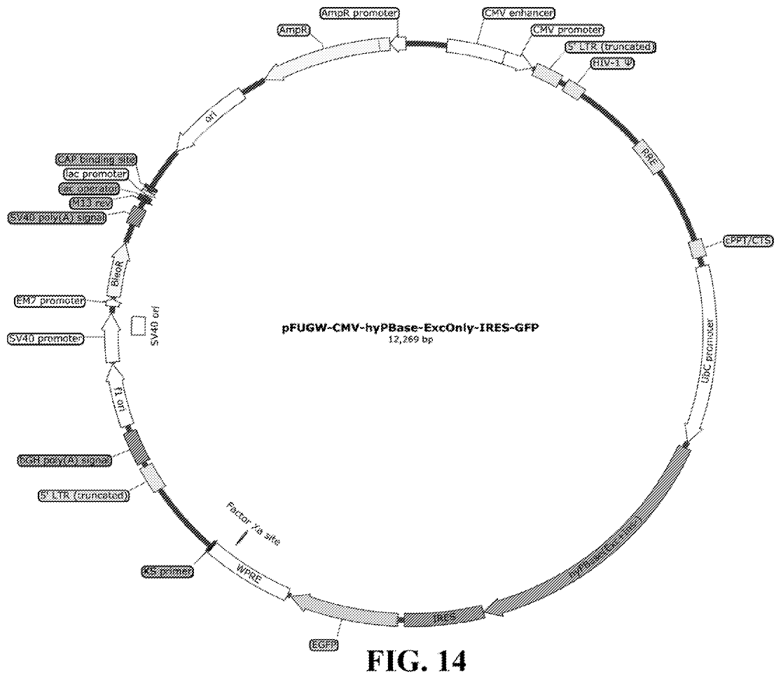

FIG. 15—Shows a flow chart for double selection base editing.

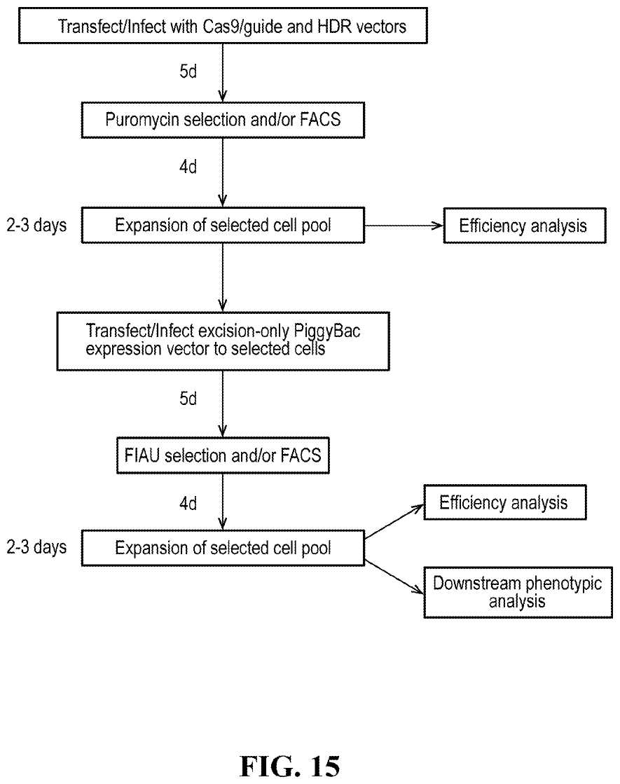

FIG. 16—Shows an overview of the CRISPR-SAVE process in accordance with certain example embodiments.

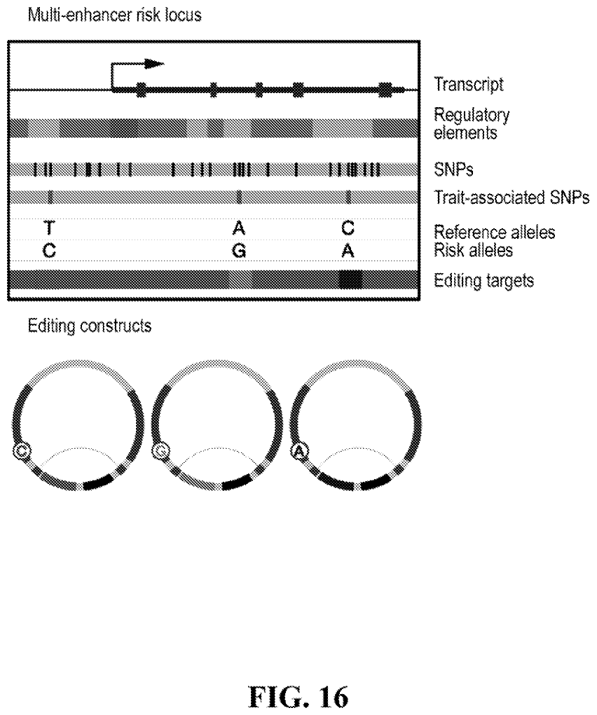

FIG. 17—Shows an overview of the process of insertion and positive selection.

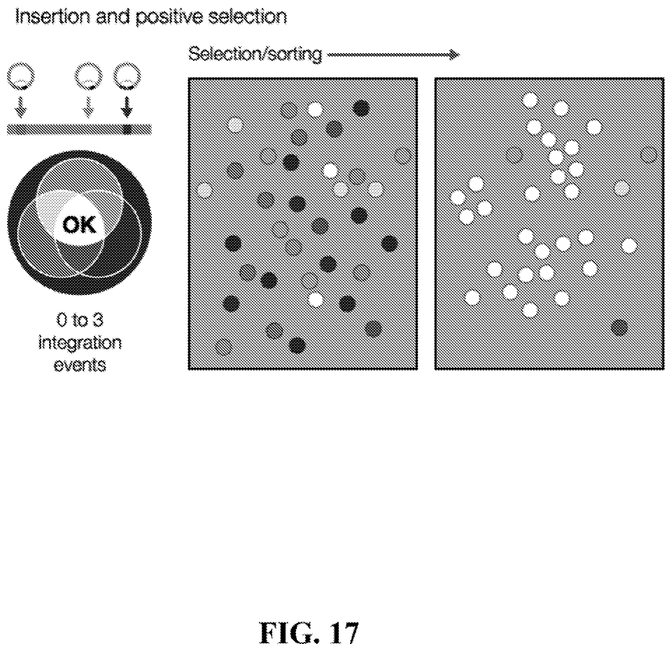

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton el al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a,” “an,” and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

Reference throughout this specification to “one embodiment,” “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited in this application may be indicative of the level of skill in the art(s) to which the application pertains. All publications, published patent documents, and patent applications cited herein are hereby incorporated by reference to the same extent as though each individual publication, published patent document, or patent application was specifically and individually indicated as being incorporated by reference.

### Overview

Embodiments disclosed herein are directed to constructs, systems, and methods for screening genetic variants to identify causal variants of a given cellular phenotype, such as a particular disease phenotype. Many genetic variants may be correlated with a given phenotype but only a subset of those genetic variants, or even a single variant in certain instances, may be the causal variant driving the phenotype. Thus, the embodiments disclosed herein provide a way to screen one or more variants to identify causal variants for a particular cellular and/or organismal phenotype. Existing methods and systems suffer from low efficiency, e.g., are time consuming, lack scalability and reproducibility, and therefore may take a year or more to complete a screen. The embodiments disclosed herein provide improved editing efficiency that is “scarless”; that is, unintended secondary edits or markers that may impact the observed phenotype are not left behind, or few unintended confounding modifications are left behind. In other embodiments, no scar is left behind from selection. Likewise, the embodiments disclosed herein allow for higher throughput through the use of modular cloning and simple and rapid efficiency determination. In particular, the embodiments disclosed herein may be useful in screening for causal variants in both coding and non-coding regions of a genome.

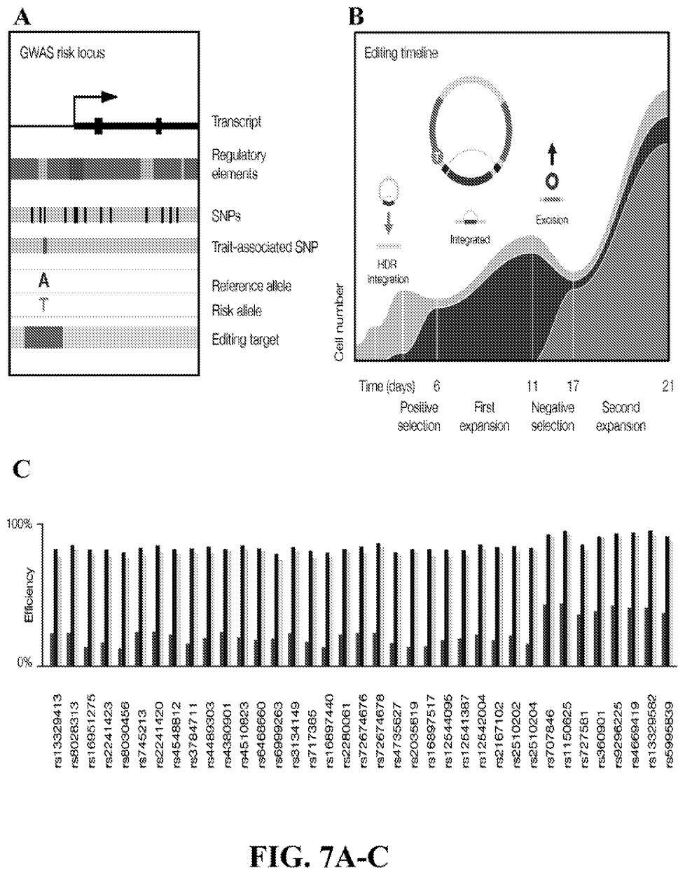

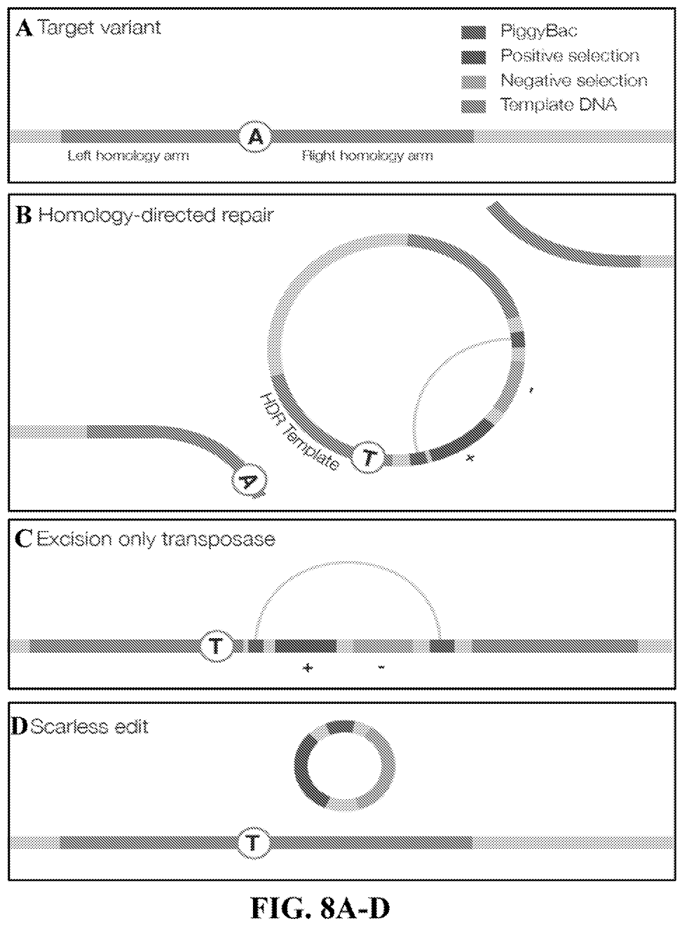

In general, the screening systems disclosed herein comprise a gene-editing system and/or a nucleotide sequence encoding the gene-editing system, and a homology-directed repair construct. The HDR repair construct encodes the gene edit to be screened and a double selection cassette. In certain example embodiments, the gene-editing system is a CRISPR-based gene editing system. The HDR constructs are modular in nature allowing for the high throughput screening of multiple variants. The HDR construct backbone may be cloned into a suitable delivery vector. In certain embodiments, the target sequence may be in a coding or non-coding region of a genome. In certain example embodiments, the gene-editing system is a homology-directed repair (HDR) system. In certain example embodiments, the gene-editing system is a CRISPR gene editing system. The targeted gene edits are encoded on a HDR construct. The design of the HDR construct allows for modular cloning to facilitate higher throughput screening of variants. The HDR construct further provides two selection cassettes, which both facilitate rapid efficiency determination, as well as allow for selection of seamless or scarless edits that do not leave behind unwanted artifacts that may otherwise effect the observed phenotype. An overview of the editing process, referred to herein as CRISPR-SAVE (Scalable Accurate Variant Editing) is provided in FIGS. 7 and 8.

### Homology-Directed Constructs

In certain example embodiments, the HDR construct comprises a left and right homology arm, and an excisable double selection cassette located within the left and right homology arms. The left and right homology arms provide a degree of complementarity to the target region comprising a target locus into which the genetic edit is to be introduced. The genetic edit may be encoded in either the left or right homology arm. The double selection cassette may encompass a first selection marker and a second selection marker. The first and second selection mark may be located between a first and second excision site.

As used herein, a “target sequence” or “target locus” is intended to designate either one target sequence or more than one target sequence, i.e. any sequence of interest at which a genomic edit or analysis is aimed. In some embodiments, a target sequence as described herein may be a target locus, a region of the genome into which a genomic edit is to be inserted. Thus, the sample may comprise more than one target sequence or “target locus” or a plurality of target sequences or target loci as desired for the particular application. A target sequence or locus may be a nucleotide sequence, particularly a specific sequence at the target locus for incorporation of the desired nucleic acid edit. The nucleotide sequence may be a DNA sequence, a RNA sequence or a mixture thereof. The target locus may be in a coding or non-coding region of a nucleic acid sequence.

### A. Homology Arms

An HDR construct as described herein may be used to introduce specific nucleic acid sequences, such as a single nucleotide variant, into a genome or a nucleic acid sequence. Conversely, such constructs may be used to insert the correct nucleotide sequence into an existing variant nucleic acid such that the resulting nucleic acid lacks the variation. Such constructs may in some embodiments be used to insert new elements into a gene that were not previously present. In order for such constructs to work, a certain amount of homology surrounding the target sequence is necessary in order to achieve homologous recombination between the nucleic acid introduced into the cell and the native nucleic acid of the cell at the target insertion site. As used herein, a “homology arm” refers to a region or segment of the genome on one or both sides of the target site whose DNA sequence is identical to the target genome sequence such that homologous recombination can occur between, resulting in insertion of the desired nucleic acid into the target site and/or removal of the equivalent nucleic acid from the native genome or nucleic acid. A homology arm may be any distance from the target site, as long as the activity of the transposase is not affected. For example, an insertion or target site may generally be about 100 bp or less from the target site, or may be less than 10 bp away, such as 100 bp, 95 bp, 90 bp, 85 bp, 80 bp, 75 bp, 70 bp, 65 bp, 60 bp, 55 bp, 50 bp, 45 bp, 40 bp, 35 bp, 30 bp, 25 bp, 20 bp, 15 bp, 10 bp, 5 bp, 4 bp, 3 bp, 2 bp, or 1 bp.

Efficiency of the HDR construct may be influenced by the overall length of the homology arm(s), with larger homology arms, i.e., up to about 200 bp may be beneficial in some embodiments, or shorter homology arms may provide more desirable results in some embodiments, for example as short as a few base pairs. Therefore, in some embodiments, a homology arm as described herein may be 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, 1000 or more base pairs in length. In some embodiments, the left and right homology arms may each be from about 700 bp to about 1000 bp. In some embodiments, one or more repetitive DNA sequence(s) may be present or incorporated into a homology arm as described herein.

One or both homology arms as described herein can encode a genomic edit to be incorporated at a target locus. As used herein, a “genomic edit” or “edit” refers to a particular nucleotide or nucleic acid sequence to be inserted into a target locus. A genomic edit may be incorporated into a construct as described herein, for example into a homology arm. An edit may be engineered or incorporated into either the left or right homology arm such that the homology arm encodes the genomic edit. The genomic edit may introduce one or more variant sequences or a locus, i.e., a sequence that differences from a wild type sequence at a locus or it otherwise recognized as the standard sequence at a given locus for a given population or sub-population of cells or organisms. Alternatively, the genomic edit may restore the wild type sequence at a given locus.

### B. Double Selection Cassette

A construct as described herein may contain an excisable double-selection cassette. Such a cassette is, in some embodiments, located within or between the right and left homology arms. A double selection cassette in accordance with the invention may comprise a first and a second selection marker. In some embodiments, the first and second selection markers are located between a first and a second excision site. In some embodiments, the first and/or second excision site may be a transposase recognition site, a restriction site, or the like. One of skill in the art will understand and be readily able to identify and use one or more excision sites as appropriate for the particular application or use.

In some embodiments, a construct or vector as described herein may have one or more selection markers. As used herein, a “selection marker” or “selectable marker” refers to a genetic element that confers a trait that may be used to differentiate those cells into which the construct or vector has been introduced and/or removed. In some embodiments, the first selection marker is a drug resistance gene, and the second selection marker is a drug sensitivity gene.

In accordance with the invention, the first selection marker is a positive selection marker. Positive selection will enable identification and/or selection of those cells into which the HDR construct has been incorporated. In some embodiments, a positive selection marker may be a drug resistance gene, such as an antibiotic resistance gene. Antibiotic resistance genes used in this way result in those cells that receive the HDR construct being able to survive exposure to a particular drug or antibiotic, thus identifying cells into which the HDR construct was successfully incorporated. Drug resistance genes are well known in the art and may include any gene appropriate for use with the invention, including, but not limited to, zeocin, blasticidin, geneticin (G-418), hygromycin B, puromycin, cytosine deaminase, rifampin, acriflavin, ampicillin, beta-lactamase, bacitracin, blastocidin, bleomycin, carbenicillin, cephalosporin, coumarin, daunorubicin, doxicycline, doxorubicin, penicillin, kanamycin, erythromycin, fosfomycin, gancyclovir, gentamicin, hygromycin, mupirocin, spectinomycin, streptomycin, tetracycline, triclosan, tunicamycin, vancomycin, xipamide, or any others appropriate in accordance with the invention.

In some embodiments, the second selection marker is a negative selection marker and will enable identification/elimination of any cells that retain the double selection cassette following removal of the cassette. Any negative selection marker may be used as appropriate, including, but not limited to, thymidine kinase (TK), URA3, HPRT/gpt, codA, hygromycin phosphotransferase, or any combinations thereof. In some embodiments, negative selection in plant cells may involve the use of NPT II, hygromycin B phosphotransferase (hpt), phosphinothricin N-acetyltransferase (PAT), or any others that may be appropriate for use with the invention. In other embodiments, other site-specific recombinase-mediated excision of a marker gene may be used for removal of the double selection cassette, either in addition to, or instead of, removal as described herein, if appropriate, such as the Cre/LoxP, FLP/FRT, or R-RS systems.

In some embodiments, the first or the second selection marker may be operably linked to a promoter for expression in the cell into which the gene is inserted. In other embodiments, both selection markers are operably linked to separate promoters for expression in a cell. In some embodiments, the elements of a HDR construct as described herein may be present on a single nucleic acid construct or a single vector. In other embodiments, such elements may be present on more than one construct or vector. In some embodiments, a HDR construct as described herein may further comprise a screenable marker, such as green fluorescent protein (GFP), blue-white screening (lacZ) β-glucuronidase (GUS), luciferase (LUC), firefly luciferase (ff-LUC). Fluorescent markers as described herein may be used for fluorescence-activated cell sorting (FACS) in order to achieve isolation of positive cell pools, wherein the fluorescent marker comprises Blue-TagBFP, Cyan-Cerulean, Green-Tag GFP2, Yellow-YPet, Red-TagRFP, Far Red-mKate2.

Briefly, an HDR construct as described herein will bind to a target locus with the right and left homology arms and, as a result of homologous recombination, transfer any/all elements present between the right and left homology arms into the target locus of the cell, i.e., the destination genetic locus or genome. This will replace the genetic information at the target locus with the genetic information present on the HDR construct. A positive selection may then be performed in order to eliminate any cells that have not received a copy of the HDR construct and will therefore lack the necessary gene to survive the selection. The double selection cassette is then removed or excised using a transposase as described herein, and a subsequent negative selection step is then performed in order to eliminate any cells that retained the double selection cassette following excision/removal.

As used herein, a “reference genomic sequence” is intended to encompass the singular and the plural. As such, when referring to a reference sequence, cases in which more than one reference sequence is available are also contemplated. Preferably, the reference sequence is a plurality of reference sequences, the number of which may be over 30; 50; 70; 100; 200; 300; 500; 1,000 and above. In certain example embodiments, the reference sequence is a genomic sequence. In certain example embodiments, the reference sequence is a plurality of genomic sequences. In certain example embodiments, the reference sequence is a plurality of genomic sequences from the same species. In certain other example embodiments, the reference sequence is a plurality of genomic sequences from different species.

### Homology-Directed Repair Vectors

The HDR constructs may be cloned into a delivery vector. In certain example embodiments, the backbone of such a vector enables uniform, one-step assembly for incorporation of homology arms. In some embodiments, a HDR vector of the invention is a transformation delivery vector, an expression vector, a cloning vector, a recombinant vector. In specific embodiments, a HDR vector may be a viral delivery vector. A vector in accordance with the invention may be a viral delivery vector, including, but not limited to, a lentiviral vector, RNP, Murine Leukemia Virus (MuLV), Human Immunodeficiency Virus (HIV), Human T-cell Lymphotrophic Virus (HTLV), linearized plasmid, non-integrating lentivirus, SV40 virus, retroviruses, gamma retrovirus, adenovirus, adeno-associated virus, herpes simplex virus (HSV), Vaccinia virus, or an oncoretrovirus. A vector or construct of the invention may also be delivered to a target cell using liposomes, dendrimers, cationic polymers, magnet-mediated transfection, electroporation, biolistic particles, microinjection, laserfection/optoinjection, or any other that may be appropriate for use with the invention.

In certain aspects, a HDR vector as described herein, e.g., for delivering or introducing into a cell a HDR construct as described herein, may also have additional elements. As used herein, a “vector” is a tool that allows or facilitates the transfer of an entity from one environment to another. It is a replicon, such as a plasmid, phage, cosmid, or artificial chromosome, into which another DNA segment may be inserted so as to bring about the replication of the inserted segment. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular, double-stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication-defective retroviruses, adenoviruses, replication defective adenoviruses, and adeno-associated viruses (AAVs)). Viral vectors may also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors may comprise a construct of the invention in a form suitable for expression of a nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). With regard to recombination and cloning methods, mention is made of U.S. patent application Ser. No. 10/815,730, published Sep. 2, 2004 as US 2004/0171156 A1, the contents of which are herein incorporated by reference in their entirety. Thus, the embodiments disclosed herein may also comprise transgenic cells comprising a construct as described herein. Such a construct may comprise a CRISPR effector system. In certain example embodiments, the transgenic cell may function as an individual discrete volume. In other words, samples comprising a masking construct may be delivered to a cell, for example in a suitable delivery vesicle and if the target is present in the delivery vesicle the CRISPR effector is activated and a detectable signal generated.

### Gene Editing Systems

As described herein, the present invention provides constructs, vectors, and related methods for directed, specific genomic repair, wherein one or more nucleotides may be edited or corrected, or any desired number of bases may be edited using a gene editing system. Gene editing as described herein is based on homologous recombination between a HDR construct of the invention and the target locus. As noted above, the HDR construct may be optionally delivered using a delivery vector.

Also with respect to general information on gene editing systems that may be used in the present invention, mention is made of the following
Multiplex genome engineering using CRISPR/Cas systems. Cong, L., Ran, F. A., Cox, D., Lin, S., Barretto, R., Habib, N., Hsu, P. D., Wu, X., Jiang, W., Marraffini, L. A., & Zhang, F. Science February 15; 339(6121):819-23 (2013);
RNA-guided editing of bacterial genomes using CRISPR-Cas systems. Jiang W., Bikard D., Cox D., Zhang F, Marraffini L A. Nat Biotechnol March; 31(3):233-9 (2013);
One-Step Generation of Mice Carrying Mutations in Multiple Genes by CRISPR/Cas-Mediated Genome Engineering. Wang H., Yang H., Shivalila C S., Dawlaty M M., Cheng A W., Zhang F., Jaenisch R. Cell May 9; 153(4):910-8 (2013);
Optical control of mammalian endogenous transcription and epigenetic states. Konermann S, Brigham M D, Trevino A E, Hsu P D, Heidenreich M, Cong L, Platt R J, Scott D A, Church G M, Zhang F. Nature. August 22; 500(7463):472-6. doi: 10.1038/Nature12466. Epub 2013 Aug. 23 (2013);
Double Nicking by RNA-Guided CRISPR Cas9 for Enhanced Genome Editing Specificity. Ran, F A., Hsu, P D., Lin, C Y., Gootenberg, J S., Konermann, S., Trevino, A E., Scott, D A., Inoue, A., Matoba, S., Zhang, Y., & Zhang, F. Cell August 28. pii: S0092-8674(13)01015-5 (2013-A);
DNA targeting specificity of RNA-guided Cas9 nucleases. Hsu, P., Scott, D., Weinstein, J., Ran, F A., Konermann, S., Agarwala, V., Li, Y., Fine, E., Wu, X., Shalem, O., Cradick, T J., Marraffini, L A., Bao, G., & Zhang, F. Nat Biotechnol doi:10.1038/nbt.2647 (2013);
Genome engineering using the CRISPR-Cas9 system. Ran, F A., Hsu, P D., Wright, J., Agarwala, V., Scott, D A., Zhang, F. Nature Protocols November; 8(11):2281-308 (2013-B);
Genome-Scale CRISPR-Cas9 Knockout Screening in Human Cells. Shalem, O., Sanjana, N E., Hartenian, E., Shi, X., Scott, D A., Mikkelson, T., Heckl, D., Ebert, B L., Root, D E., Doench, J G., Zhang, F. Science December 12. (2013). [Epub ahead of print];
Crystal structure of cas9 in complex with guide RNA and target DNA. Nishimasu, H., Ran, F A., Hsu, P D., Konermann, S., Shehata, S I., Dohmae, N., Ishitani, R., Zhang, F., Nureki, O. Cell February 27, 156(5):935-49 (2014);
Genome-wide binding of the CRISPR endonuclease Cas9 in mammalian cells. Wu X., Scott D A., Kriz A J., Chiu A C., Hsu P D., Dadon D B., Cheng A W., Trevino A E., Konermann S., Chen S., Jaenisch R., Zhang F., Sharp P A. Nat Biotechnol. April 20. doi: 10.1038/nbt.2889 (2014);
CRISPR-Cas9 Knockin Mice for Genome Editing and Cancer Modeling. Platt R J, Chen S, Zhou Y, Yim M J, Swiech L, Kempton H R, Dahlman J E, Parnas O, Eisenhaure T M, Jovanovic M, Graham D B, Jhunjhunwala S, Heidenreich M, Xavier R J, Langer R, Anderson D G, Hacohen N, Regev A, Feng G, Sharp P A, Zhang F. Cell 159(2): 440-455 DOI: 10.1016/j.cell.2014.09.014(2014);
Development and Applications of CRISPR-Cas9 for Genome Engineering, Hsu P D, Lander E S, Zhang F., Cell. June 5; 157(6):1262-78 (2014).
Genetic screens in human cells using the CRISPR/Cas9 system, Wang T, Wei J J, Sabatini D M, Lander E S., Science. January 3; 343(6166): 80-84. doi:10.1126/science.1246981 (2014);
Rational design of highly active sgRNAs for CRISPR-Cas9-mediated gene inactivation, Doench J G, Hartenian E, Graham D B, Tothova Z, Hegde M, Smith I, Sullender M, Ebert B L, Xavier R J, Root D E., (published online 3 Sep. 2014) Nat Biotechnol. December; 32(12):1262-7 (2014);
In vivo interrogation of gene function in the mammalian brain using CRISPR-Cas9, Swiech L, Heidenreich M, Banerjee A, Habib N, Li Y, Trombetta J, Sur M, Zhang F., (published online 19 Oct. 2014) Nat Biotechnol. January; 33(1):102-6 (2015);
Genome-scale transcriptional activation by an engineered CRISPR-Cas9 complex, Konermann S, Brigham M D, Trevino A E, Joung J, Abudayyeh O O, Barcena C, Hsu P D, Habib N, Gootenberg J S, Nishimasu H, Nureki O, Zhang F., Nature. January 29; 517(7536):583-8 (2015).
A split-Cas9 architecture for inducible genome editing and transcription modulation, Zetsche B, Volz S E, Zhang F., (published online 2 Feb. 2015) Nat Biotechnol. February; 33(2):139-42 (2015);
Genome-wide CRISPR Screen in a Mouse Model of Tumor Growth and Metastasis, Chen S, Sanjana N E, Zheng K, Shalem O, Lee K, Shi X, Scott D A, Song J, Pan J Q, Weissleder R, Lee H, Zhang F, Sharp P A. Cell 160, 1246-1260, Mar. 12, 2015 (multiplex screen in mouse), and
In vivo genome editing using Staphylococcus aureus Cas9, Ran F A, Cong L, Yan W X, Scott D A, Gootenberg J S, Kriz A J, Zetsche B, Shalem O, Wu X, Makarova K S, Koonin E V, Sharp P A, Zhang F., (published online 1 Apr. 2015), Nature. April 9; 520(7546): 186-91 (2015).
Shalem et al., “High-throughput functional genomics using CRISPR-Cas9,” Nature Reviews Genetics 16, 299-311 (May 2015).
Xu et al., “Sequence determinants of improved CRISPR sgRNA design,” Genome Research 25, 1147-1157 (August 2015).
Parnas et al., “A Genome-wide CRISPR Screen in Primary Immune Cells to Dissect Regulatory Networks,” Cell 162, 675-686 (Jul. 30, 2015).
Ramanan et al., CRISPR/Cas9 cleavage of viral DNA efficiently suppresses hepatitis B virus,” Scientific Reports 5:10833. doi: 10.1038/srep10833 (Jun. 2, 2015)
Nishimasu et al., Crystal Structure of Staphylococcus aureus Cas9,” Cell 162, 1113-1126 (Aug. 27, 2015)
Zetsche et al., “Cpf1 Is a Single RNA-Guided Endonuclease of a Class 2 CRISPR-Cas System, ” Cell 163, 1-13 (Oct. 22, 2015)
Shmakov et al., “Discovery and Functional Characterization of Diverse Class 2 CRISPR-Cas Systems,” Molecular Cell 60, 1-13 (Available online Oct. 22, 2015)
   each of which is incorporated herein by reference, may be considered in the practice of the instant invention, and discussed briefly below:
Cong et al. engineered type II CRISPR-Cas systems for use in eukaryotic cells based on both Streptococcus thermophilus Cas9 and also Streptococcus pyogenes Cas9 and demonstrated that Cas9 nucleases can be directed by short RNAs to induce precise cleavage of DNA in human and mouse cells. Their study further showed that Cas9 as converted into a nicking enzyme can be used to facilitate homology-directed repair in eukaryotic cells with minimal mutagenic activity. Additionally, their study demonstrated that multiple guide sequences can be encoded into a single CRISPR array to enable simultaneous editing of several at endogenous genomic loci sites within the mammalian genome, demonstrating easy programmability and wide applicability of the RNA-guided nuclease technology. This ability to use RNA to program sequence specific DNA cleavage in cells defined a new class of genome engineering tools. These studies further showed that other CRISPR loci are likely to be transplantable into mammalian cells and can also mediate mammalian genome cleavage. Importantly, it can be envisaged that several aspects of the CRISPR-Cas system can be further improved to increase its efficiency and versatility.
Jiang et al. used the clustered, regularly interspaced, short palindromic repeats (CRISPR)-associated Cas9 endonuclease complexed with dual-RNAs to introduce precise mutations in the genomes of Streptococcus pneumoniae and Escherichia coli. The approach relied on dual-RNA:Cas9-directed cleavage at the targeted genomic site to kill unmutated cells and circumvents the need for selectable markers or counter-selection systems. The study reported reprogramming dual-RNA:Cas9 specificity by changing the sequence of short CRISPR RNA (crRNA) to make single- and multinucleotide changes carried on editing templates. The study showed that simultaneous use of two crRNAs enabled multiplex mutagenesis. Furthermore, when the approach was used in combination with recombineering, in S. pneumoniae, nearly 100% of cells that were recovered using the described approach contained the desired mutation, and in E. coli, 65% that were recovered contained the mutation.
Wang et al. (2013) used the CRISPR/Cas system for the one-step generation of mice carrying mutations in multiple genes which were traditionally generated in multiple steps by sequential recombination in embryonic stem cells and/or time-consuming intercrossing of mice with a single mutation. The CRISPR/Cas system will greatly accelerate the in vivo study of functionally redundant genes and of epistatic gene interactions.
Konermann et al. (2013) addressed the need in the art for versatile and robust technologies that enable optical and chemical modulation of DNA-binding domains based CRISPR Cas9 enzyme and also Transcriptional Activator Like Effectors
Ran et al. (2013-A) described an approach that combined a Cas9 nickase mutant with paired guide RNAs to introduce targeted double-strand breaks. This addresses the issue of the Cas9 nuclease from the microbial CRISPR-Cas system being targeted to specific genomic loci by a guide sequence, which can tolerate certain mismatches to the DNA target and thereby promote undesired off-target mutagenesis. Because individual nicks in the genome are repaired with high fidelity, simultaneous nicking via appropriately offset guide RNAs is required for double-stranded breaks and extends the number of specifically recognized bases for target cleavage. The authors demonstrated that using paired nicking can reduce off-target activity by 50- to 1,500-fold in cell lines and to facilitate gene knockout in mouse zygotes without sacrificing on-target cleavage efficiency. This versatile strategy enables a wide variety of genome editing applications that require high specificity.
Hsu et al. (2013) characterized SpCas9 targeting specificity in human cells to inform the selection of target sites and avoid off-target effects. The study evaluated >700 guide RNA variants and SpCas9-induced indel mutation levels at >100 predicted genomic off-target loci in 293T and 293FT cells. The authors that SpCas9 tolerates mismatches between guide RNA and target DNA at different positions in a sequence-dependent manner, sensitive to the number, position and distribution of mismatches. The authors further showed that SpCas9-mediated cleavage is unaffected by DNA methylation and that the dosage of SpCas9 and sgRNA can be titrated to minimize off-target modification. Additionally, to facilitate mammalian genome engineering applications, the authors reported providing a web-based software tool to guide the selection and validation of target sequences as well as off-target analyses.
Ran et al. (2013-B) described a set of tools for Cas9-mediated genome editing via non-homologous end joining (NHEJ) or homology-directed repair (HDR) in mammalian cells, as well as generation of modified cell lines for downstream functional studies. To minimize off-target cleavage, the authors further described a double-nicking strategy using the Cas9 nickase mutant with paired guide RNAs. The protocol provided by the authors experimentally derived guidelines for the selection of target sites, evaluation of cleavage efficiency and analysis of off-target activity. The studies showed that beginning with target design, gene modifications can be achieved within as little as 1-2 weeks, and modified clonal cell lines can be derived within 2-3 weeks.
Shalem et al. described a new way to interrogate gene function on a genome-wide scale. Their studies showed that delivery of a genome-scale CRISPR-Cas9 knockout (GeCKO) library targeted 18,080 genes with 64,751 unique guide sequences enabled both negative and positive selection screening in human cells. First, the authors showed use of the GeCKO library to identify genes essential for cell viability in cancer and pluripotent stem cells. Next, in a melanoma model, the authors screened for genes whose loss is involved in resistance to vemurafenib, a therapeutic that inhibits mutant protein kinase BRAF. Their studies showed that the highest-ranking candidates included previously validated genes NF1 and MED12 as well as novel hits NF2, CUL3, TADA2B, and TADA1. The authors observed a high level of consistency between independent guide RNAs targeting the same gene and a high rate of hit confirmation, and thus demonstrated the promise of genome-scale screening with Cas9.
Nishimasu et al. reported the crystal structure of Streptococcus pyogenes Cas9 in complex with sgRNA and its target DNA at 2.5 A° resolution. The structure revealed a bilobed architecture composed of target recognition and nuclease lobes, accommodating the sgRNA:DNA heteroduplex in a positively charged groove at their interface. Whereas the recognition lobe is essential for binding sgRNA and DNA, the nuclease lobe contains the HNH and RuvC nuclease domains, which are properly positioned for cleavage of the complementary and non-complementary strands of the target DNA, respectively. The nuclease lobe also contains a carboxyl-terminal domain responsible for the interaction with the protospacer adjacent motif (PAM). This high-resolution structure and accompanying functional analyses have revealed the molecular mechanism of RNA-guided DNA targeting by Cas9, thus paving the way for the rational design of new, versatile genome-editing technologies.
Wu et al. mapped genome-wide binding sites of a catalytically inactive Cas9 (dCas9) from Streptococcus pyogenes loaded with single guide RNAs (sgRNAs) in mouse embryonic stem cells (mESCs). The authors showed that each of the four sgRNAs tested targets dCas9 to between tens and thousands of genomic sites, frequently characterized by a 5-nucleotide seed region in the sgRNA and an NGG protospacer adjacent motif (PAM). Chromatin inaccessibility decreases dCas9 binding to other sites with matching seed sequences; thus 70% of off-target sites are associated with genes. The authors showed that targeted sequencing of 295 dCas9 binding sites in mESCs transfected with catalytically active Cas9 identified only one site mutated above background levels. The authors proposed a two-state model for Cas9 binding and cleavage, in which a seed match triggers binding but extensive pairing with target DNA is required for cleavage.
Platt et al. established a Cre-dependent Cas9 knockin mouse. The authors demonstrated in vivo as well as ex vivo genome editing using adeno-associated virus (AAV)-, lentivirus-, or particle-mediated delivery of guide RNA in neurons, immune cells, and endothelial cells.
Hsu et al. (2014) is a review article that discusses generally CRISPR-Cas9 history from yogurt to genome editing, including genetic screening of cells.
Wang et al. (2014) relates to a pooled, loss-of-function genetic screening approach suitable for both positive and negative selection that uses a genome-scale lentiviral single guide RNA (sgRNA) library.
Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.
Swiech et al. demonstrate that AAV-mediated SpCas9 genome editing can enable reverse genetic studies of gene function in the brain.
Konermann et al. (2015) discusses the ability to attach multiple effector domains, e.g., transcriptional activator, functional and epigenomic regulators at appropriate positions on the guide such as stem or tetraloop with and without linkers.
Zetsche et al. demonstrates that the Cas9 enzyme can be split into two and hence the assembly of Cas9 for activation can be controlled.
Chen et al. relates to multiplex screening by demonstrating that a genome-wide in vivo CRISPR-Cas9 screen in mice reveals genes regulating lung metastasis.
Ran et al. (2015) relates to SaCas9 and its ability to edit genomes and demonstrates that one cannot extrapolate from biochemical assays.
Shalem et al. (2015) described ways in which catalytically inactive Cas9 (dCas9) fusions are used to synthetically repress (CRISPRi) or activate (CRISPRa) expression, showing. advances using Cas9 for genome-scale screens, including arrayed and pooled screens, knockout approaches that inactivate genomic loci and strategies that modulate transcriptional activity.
Xu et al. (2015) assessed the DNA sequence features that contribute to single guide RNA (sgRNA) efficiency in CRISPR-based screens. The authors explored efficiency of CRISPR/Cas9 knockout and nucleotide preference at the cleavage site. The authors also found that the sequence preference for CRISPRi/a is substantially different from that for CRISPR/Cas9 knockout.
Parnas et al. (2015) introduced genome-wide pooled CRISPR-Cas9 libraries into dendritic cells (DCs) to identify genes that control the induction of tumor necrosis factor (Tnf) by bacterial lipopolysaccharide (LPS). Known regulators of Tlr4 signaling and previously unknown candidates were identified and classified into three functional modules with distinct effects on the canonical responses to LPS.
Ramanan et al (2015) demonstrated cleavage of viral episomal DNA (cccDNA) in infected cells. The HBV genome exists in the nuclei of infected hepatocytes as a 3.2 kb double-stranded episomal DNA species called covalently closed circular DNA (cccDNA), which is a key component in the HBV life cycle whose replication is not inhibited by current therapies. The authors showed that sgRNAs specifically targeting highly conserved regions of HBV robustly suppresses viral replication and depleted cccDNA.
Nishimasu et al. (2015) reported the crystal structures of SaCas9 in complex with a single guide RNA (sgRNA) and its double-stranded DNA targets, containing the 5′-TTGAAT-3′ PAM and the 5′-TTGGGT-3′ PAM. A structural comparison of SaCas9 with SpCas9 highlighted both structural conservation and divergence, explaining their distinct PAM specificities and orthologous sgRNA recognition.
Zetsche et al. (2015) reported the characterization of Cpf1, a putative class 2 CRISPR effector. It was demonstrated that Cpf1 mediates robust DNA interference with features distinct from Cas9. Identifying this mechanism of interference broadens our understanding of CRISPR-Cas systems and advances their genome editing applications.
Shmakov et al. (2015) reported the characterization of three distinct Class 2 CRISPR-Cas systems. The effectors of two of the identified systems, C2c1 and C2c3, contain RuvC like endonuclease domains distantly related to Cpf1. The third system, C2c2, contains an effector with two predicted HEPN RNase domains.

Also, “Dimeric CRISPR RNA-guided FokI nucleases for highly specific genome editing”, Shengdar Q. Tsai, Nicolas Wyvekens, Cyd Khayter, Jennifer A. Foden, Vishal Thapar, Deepak Reyon, Mathew J. Goodwin, Martin J. Aryee, J. Keith Joung Nature Biotechnology 32(6): 569-77 (2014), relates to dimeric RNA-guided FokI Nucleases that recognize extended sequences and can edit endogenous genes with high efficiencies in human cells.

One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms.

In advantageous embodiments of the invention, the methods provided herein use isolated, non-naturally occurring, recombinant or engineered DNA binding proteins that comprise TALE monomers or TALE monomers or half monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, “TALE monomers” or “monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X1-11-(X12X13)-X14-33 or 34 or 35, where the subscript indicates the amino acid position and X represents any amino acid. X12X13 indicate the RVDs. In some polypeptide monomers, the variable amino acid at position 13 is missing or absent and in such monomers, the RVD consists of a single amino acid. In such cases the RVD may be alternatively represented as X*, where X represents X12 and (*) indicates that X13 is absent. The DNA binding domain comprises several repeats of TALE monomers and this may be represented as (X1-11-(X12X13)-X14-33 or 34 or 35)z, where in an advantageous embodiment, z is at least 5 to 40. In a further advantageous embodiment, z is at least 10 to 26.

The TALE monomers have a nucleotide binding affinity that is determined by the identity of the amino acids in its RVD. For example, polypeptide monomers with an RVD of NI preferentially bind to adenine (A), monomers with an RVD of NG preferentially bind to thymine (T), monomers with an RVD of HD preferentially bind to cytosine (C) and monomers with an RVD of NN preferentially bind to both adenine (A) and guanine (G). In yet another embodiment of the invention, monomers with an RVD of IG preferentially bind to T. Thus, the number and order of the polypeptide monomer repeats in the nucleic acid binding domain of a TALE determines its nucleic acid target specificity. In still further embodiments of the invention, monomers with an RVD of NS recognize all four base pairs and may bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011), each of which is incorporated by reference in its entirety.

The polypeptides used in methods of the invention are isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In a preferred embodiment of the invention, polypeptide monomers having RVDs RN, NN, NK, SN, NH, KN, HN, NQ, HH, RG, KH, RH and SS preferentially bind to guanine. In a much more advantageous embodiment of the invention, polypeptide monomers having RVDs RN, NK, NQ, HH, KH, RH, SS and SN preferentially bind to guanine and thereby allow the generation of TALE polypeptides with high binding specificity for guanine containing target nucleic acid sequences. In an even more advantageous embodiment of the invention, polypeptide monomers having RVDs HH, KH, NH, NK, NQ, RH, RN and SS preferentially bind to guanine and thereby allow the generation of TALE polypeptides with high binding specificity for guanine containing target nucleic acid sequences. In a further advantageous embodiment, the RVDs that have high binding specificity for guanine are RN, NH RH and KH. Furthermore, polypeptide monomers having an RVD of NV preferentially bind to adenine and guanine. In more preferred embodiments of the invention, monomers having RVDs of H*, HA, KA, N*, NA, NC, NS, RA, and S* bind to adenine, guanine, cytosine and thymine with comparable affinity.

The predetermined N-terminal to C-terminal order of the one or more polypeptide monomers of the nucleic acid or DNA binding domain determines the corresponding predetermined target nucleic acid sequence to which the polypeptides of the invention will bind. As used herein the monomers and at least one or more half monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases, this region may be referred to as repeat 0. In animal genomes, TALE binding sites do not necessarily have to begin with a thymine (T) and polypeptides of the invention may target DNA sequences that begin with T, A, G or C. The tandem repeat of TALE monomers always ends with a half-length repeat or a stretch of sequence that may share identity with only the first 20 amino acids of a repetitive full length TALE monomer and this half repeat may be referred to as a half-monomer. Therefore, it follows that the length of the nucleic acid or DNA being targeted is equal to the number of full monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full length capping region, while fragments that include the C-terminal 20 amino acids retain greater than 50% of the efficacy of the full length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies may be generated by any of a number of computer programs known in the art, which include but are not limited to BLAST or FASTA. Suitable computer program for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In advantageous embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Krüppel-associated box (KRAB) or fragments of the KRAB domain. In some embodiments, the effector domain is an enhancer of transcription (i.e. an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination the activities described herein.

### Excision Proteins

As used herein, an “excision protein” is a protein, or functional fragment thereof, that is involved in excision or removal of a nucleotide or nucleic acid segment. Such a protein may be an endonuclease, a transposase, or any other type of protein capable of cutting and/or excising a nucleotide or nucleic acid.

In certain example embodiments, the excision protein is a transposase. Some transposases can precisely remove any inserted nucleotides without leaving a footprint or artifact, referred to herein as a “scar.” The present invention therefore provides methods and associated constructs and vectors for scarless editing of one or more nucleotides. During transposition, the transposase recognizes transposon-specific inverted terminal repeat sequences (ITRs), i.e. excision sites, located on both ends of the double selection cassette and excises the nucleic acid from the double selection cassette. Accordingly, cells that have incorporated the HDR constructs described herein, but have not had the first and selection markers excised can be selected based on the retained presence of the second selection marker. As stated above, the second selection marker may be a negative selection marker. For example, the negative selection marker may confer drug susceptibility. Introduction of the drug to a pool of cells will remove those cells from the pool of cells from which the double selection cassette has not been excised or otherwise removed.

Various types of transposases are known and available in the art and may include, but are not limited to, an excision transposase, and/or a hyperactive transposase. In some embodiments, a transposase as described herein may comprise a mutation that alters its function. For example, certain mutations may make a particular transposase more or less active, or may result in more or less precise removal of a target sequence. In certain example embodiments, the transposase may comprise a transposase as encoded by the nucleotide sequence of SEQ ID NO:1. In some particular embodiments, a transposase as described herein may comprise a PiggyBac transposase, or a mutated version of a PiggyBac transposase. A PiggyBac transposase typically transposes nucleic acid, such as DNA, RNA, or hybrids thereof, between vectors and a target site.

### Delivery of System Components

With respect to general information delivery HDR constructs, gene editing systems, excision proteins and components of the systems described herein, including methods, materials, delivery vehicles, vectors, particles, AAV, and making and using thereof, including as to amounts and formulations, all useful in the practice of the instant invention, reference is made to: U.S. Pat. Nos. 8,999,641, 8,993,233, 8,945,839, 8,932,814, 8,906,616, 8,895,308, 8,889,418, 8,889,356, 8,871,445, 8,865,406, 8,795,965, 8,771,945 and 8,697,359; US Patent Publications US 2014-0310830 (U.S. application Ser. No. 14/105,031), US 2014-0287938 A1 (U.S. application Ser. No. 14/213,991), US 2014-0273234 A1 (U.S. application Ser. No. 14/293,674), US2014-0273232 A1 (U.S. application Ser. No. 14/290,575), US 2014-0273231 (U.S. application Ser. No. 14/259,420), US 2014-0256046 A1 (U.S. application Ser. No. 14/226,274), US 2014-0248702 A1 (U.S. application Ser. No. 14/258,458), US 2014-0242700 A1 (U.S. application Ser. No. 14/222,930), US 2014-0242699 A1 (U.S. application Ser. No. 14/183,512), US 2014-0242664 A1 (U.S. application Ser. No. 14/104,990), US 2014-0234972 A1 (U.S. application Ser. No. 14/183,471), US 2014-0227787 A1 (U.S. application Ser. No. 14/256,912), US 2014-0189896 A1 (U.S. application Ser. No. 14/105,035), US 2014-0186958 (U.S. application Ser. No. 14/105,017), US 2014-0186919 A1 (U.S. application Ser. No. 14/104,977), US 2014-0186843 A1 (U.S. application Ser. No. 14/104,900), US 2014-0179770 A1 (U.S. application Ser. No. 14/104,837) and US 2014-0179006 A1 (U.S. application Ser. No. 14/183,486), US 2014-0170753 (U.S. application Ser. No. 14/183,429); European Patents EP 2 784 162 B1 and EP 2 771 468 B1; European Patent Applications EP 2 771 468 (EP13818570.7), EP 2 764 103 (EP13824232.6), and EP 2 784 162 (EP14170383.5); and PCT Patent Publications PCT Patent Publications WO 2014/093661 (PCT/US2013/074743), WO 2014/093694 (PCT/US2013/074790), WO 2014/093595 (PCT/US2013/074611), WO 2014/093718 (PCT/US2013/074825), WO 2014/093709 (PCT/US2013/074812), WO 2014/093622 (PCT/US2013/074667), WO 2014/093635 (PCT/US2013/074691), WO 2014/093655 (PCT/US2013/074736), WO 2014/093712 (PCT/US2013/074819), W02014/093701 (PCT/US2013/074800), W02014/018423 (PCT/US2013/051418), WO 2014/204723 (PCT/US2014/041790), WO 2014/204724 (PCT/US2014/041800), WO 2014/204725 (PCT/US2014/041803), WO 2014/204726 (PCT/US2014/041804), WO 2014/204727 (PCT/US2014/041806), WO 2014/204728 (PCT/US2014/041808), WO 2014/204729 (PCT/US2014/041809).

Reference is also made to U.S. provisional patent applications 61/758,468; 61/802,174; 61/806,375; 61/814,263; 61/819,803 and 61/828,130, filed on Jan. 30, 2013; Mar. 15, 2013; Mar. 28, 2013; Apr. 20, 2013; May 6, 2013 and May 28, 2013 respectively. Reference is also made to U.S. provisional patent application 61/836,123, filed on Jun. 17, 2013. Reference is additionally made to U.S. provisional patent applications 61/835,931, 61/835,936, 61/836,127, 61/836, 101, 61/836,080 and 61/835,973, each filed Jun. 17, 2013. Further reference is made to U.S. provisional patent applications 61/862,468 and 61/862,355 filed on Aug. 5, 2013; 61/871,301 filed on Aug. 28, 2013; 61/960,777 filed on Sep. 25, 2013 and 61/961,980 filed on Oct. 28, 2013.

Reference is yet further made to: PCT Patent applications Nos: PCT/US2014/041803, PCT/US2014/041800, PCT/US2014/041809, PCT/US2014/041804 and PCT/US2014/041806, each filed Jun. 10, 2014 Jun. 10, 2014; PCT/US2014/041808 filed Jun. 11, 2014; and PCT/US2014/62558 filed Oct. 28, 2014, and U.S. Provisional Patent Applications Ser. Nos.: 61/915,150, 61/915,301, 61/915,267 and 61/915,260, each filed Dec. 12, 2013; 61/757,972 and 61/768,959, filed on Jan. 29, 2013 and Feb. 25, 2013; 61/835,936, 61/836,127, 61/836,101, 61/836,080, 61/835,973, and 61/835,931, filed Jun. 17, 2013; 62/010,888 and 62/010,879, both filed Jun. 11, 2014; 62/010,329 and 62/010,441, each filed Jun. 10, 2014; 61/939,228 and 61/939,242, each filed Feb. 12, 2014; 61/980,012, filed Apr. 15, 2014; 62/038,358, filed Aug. 17, 2014; 62/054,490, 62/055,484, 62/055,460 and 62/055,487, each filed Sep. 25, 2014; and 62/069,243, filed Oct. 27, 2014. Reference is also made to U.S. provisional patent applications Nos. 62/055,484, 62/055,460, and 62/055,487, filed Sep. 25, 2014; U.S. provisional patent application 61/980,012, filed Apr. 15, 2014; and US provisional patent application 61/939,242 filed Feb. 12, 2014.

Reference is made to PCT application designating, inter alia, the United States, application No. PCT/US14/41806, filed Jun. 10, 2014. Reference is made to U.S. provisional patent application 61/930,214 filed on Jan. 22, 2014. Reference is made to U.S. provisional patent applications 61/915,251; 61/915,260 and 61/915,267, each filed on Dec. 12, 2013. Reference is made to US provisional patent application U.S. Ser. No. 61/980,012 filed Apr. 15, 2014. Reference is made to PCT application designating, inter alia, the United States, application No. PCT/US14/41806, filed Jun. 10, 2014. Reference is made to U.S. provisional patent application 61/930,214 filed on Jan. 22, 2014. Reference is made to U.S. provisional patent applications 61/915,251; 61/915,260 and 61/915,267, each filed on Dec. 12, 2013.

Mention is also made of U.S. application Ser. No. 62/091,455, filed, 12 Dec. 2014, PROTECTED GUIDE RNAS (PGRNAS); U.S. application Ser. No. 62/096,708, 24 Dec. 2014, PROTECTED GUIDE RNAS (PGRNAS); U.S. application Ser. No. 62/091,462, 12 Dec. 2014, DEAD GUIDES FOR CRISPR TRANSCRIPTION FACTORS; U.S. application Ser. No. 62/096,324, 23 Dec. 2014, DEAD GUIDES FOR CRISPR TRANSCRIPTION FACTORS; U.S. application Ser. No. 62/091,456, 12 Dec. 2014, ESCORTED AND FUNCTIONALIZED GUIDES FOR CRISPR-CAS SYSTEMS; U.S. application Ser. No. 62/091,461, 12 Dec. 2014, DELIVERY, USE AND THERAPEUTIC APPLICATIONS OF THE CRISPR-CAS SYSTEMS AND COMPOSITIONS FOR GENOME EDITING AS TO HEMATOPOETIC STEM CELLS (HSCs); U.S. application Ser. No. 62/094,903, 19 Dec. 2014, UNBIASED IDENTIFICATION OF DOUBLE-STRAND BREAKS AND GENOMIC REARRANGEMENT BY GENOME-WISE INSERT CAPTURE SEQUENCING; U.S. application Ser. No. 62/096,761, 24 Dec. 2014, ENGINEERING OF SYSTEMS, METHODS AND OPTIMIZED ENZYME AND GUIDE SCAFFOLDS FOR SEQUENCE MANIPULATION; U.S. application Ser. No. 62/098,059, 30 Dec. 2014, RNA-TARGETING SYSTEM; U.S. application Ser. No. 62/096,656, 24 Dec. 2014, CRISPR HAVING OR ASSOCIATED WITH DESTABILIZATION DOMAINS; U.S. application Ser. No. 62/096,697, 24 Dec. 2014, CRISPR HAVING OR ASSOCIATED WITH AAV; U.S. application Ser. No. 62/098,158, 30 Dec. 2014, ENGINEERED CRISPR COMPLEX INSERTIONAL TARGETING SYSTEMS; U.S. application Ser. No. 62/151,052, 22 Apr. 2015, CELLULAR TARGETING FOR EXTRACELLULAR EXOSOMAL REPORTING; U.S. application Ser. No. 62/054,490, 24 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS; U.S. application Ser. No. 62/055,484, 25 Sep. 2014, SYSTEMS, METHODS AND COMPOSITIONS FOR SEQUENCE MANIPULATION WITH OPTIMIZED FUNCTIONAL CRISPR-CAS SYSTEMS; U.S. application Ser. No. 62/087,537, 4 Dec. 2014, SYSTEMS, METHODS AND COMPOSITIONS FOR SEQUENCE MANIPULATION WITH OPTIMIZED FUNCTIONAL CRISPR-CAS SYSTEMS; U.S. application Ser. No. 62/054,651, 24 Sep. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. application Ser. No. 62/067,886, 23 Oct. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. application Ser. No. 62/054,675, 24 Sep. 2014, DELIVERY, USE AND THERAPEUTIC APPLICATIONS OF THE CRISPR-CAS SYSTEMS AND COMPOSITIONS IN NEURONAL CELLS/TISSUES; U.S. application Ser. No. 62/054,528, 24 Sep. 2014, DELIVERY, USE AND THERAPEUTIC APPLICATIONS OF THE CRISPR-CAS SYSTEMS AND COMPOSITIONS IN IMMUNE DISEASES OR DISORDERS; U.S. application Ser. No. 62/055,454, 25 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING CELL PENETRATION PEPTIDES (CPP); U.S. application Ser. No. 62/055,460, 25 Sep. 2014, MULTIFUNCTIONAL-CRISPR COMPLEXES AND/OR OPTIMIZED ENZYME LINKED FUNCTIONAL-CRISPR COMPLEXES; U.S. application Ser. No. 62/087,475, 4 Dec. 2014, FUNCTIONAL SCREENING WITH OPTIMIZED FUNCTIONAL CRISPR-CAS SYSTEMS; U.S. application Ser. No. 62/055,487, 25 Sep. 2014, FUNCTIONAL SCREENING WITH OPTIMIZED FUNCTIONAL CRISPR-CAS SYSTEMS; U.S. application Ser. No. 62/087,546, 4 Dec. 2014, MULTIFUNCTIONAL CRISPR COMPLEXES AND/OR OPTIMIZED ENZYME LINKED FUNCTIONAL-CRISPR COMPLEXES; and U.S. application Ser. No. 62/098,285, 30 Dec. 2014, CRISPR MEDIATED IN VIVO MODELING AND GENETIC SCREENING OF TUMOR GROWTH AND METASTASIS

### Methods for Screening Variant Loci

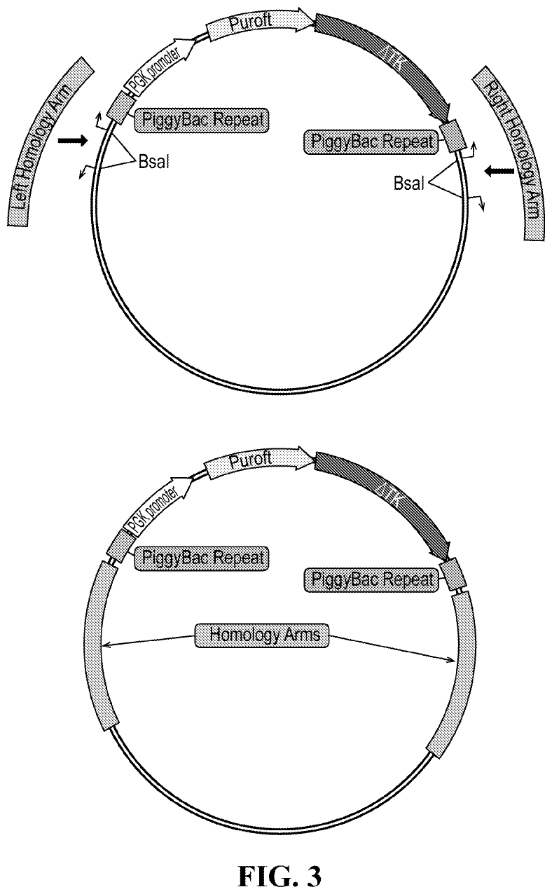

In certain aspects, the invention provides methods for variant screening in a cell or cell population. For example, the method may comprise delivering the HDR constructs described herein to one or more cells or cell populations. As noted above, HDR construct delivery may be facilitated by cloning the HDR construct into an appropriate delivery vector. In certain example embodiments, the delivery vector is a viral vector. In certain other example embodiments, the vector is a transfection vector. Example viral and transfection vectors are shown in FIG. 3. However, other suitable delivery vectors may be used as appropriate.

In some embodiments, the invention provides a method for screening one or more variant loci in a cell or a cell population into which one or more HDR constructs have been introduced. Such a system may be useful for a population of cells expressing a gene editing system that is configured to modify cellular DNA at one or more target loci. In some embodiments, a gene editing system as described herein may be a CRISPR system. In other embodiments, such a system may be useful for genomic editing of a population of cells to which a gene editing system is co-delivered along with an HDR construct or an HDR vector as described herein. A method useful as described herein for genomic editing may include steps for selection of edited cells, i.e., those cells that have incorporated the excisable double selection cassette included in an HDR construct as described herein. Such selection or identification of successfully edited cells may be accomplished with the use of a positive selection step using a first selection marker as described herein. Removal or excision of the double selection cassette may be accomplished with the use of an excision protein, such as a transposase, or with a polynucleotide encoding such an excision protein. Such a protein may be introduced to the cells in active form, along with the HDR cassette or vector, or may be included as a part of the HDR cassette or vector such that the cell expresses the nucleic acid encoding the excision protein. Once the excision protein is present and/or active in the edited cells, excision/removal of the double selection cassette can occur. Following removal of the double selection cassette, only the genomic material provided as an edit remains in the genome of the cells.

In some embodiments, those cells in which the double selection cassette has been removed may be identified and/or selected using a second selection marker. The second selection marker is a negative selection marker and will enable only those cells lacking the double selection cassette, i.e., those in which the excision protein has removed the double selection cassette, to survive. The final edited cell population will contain the edited nucleic acid, and will lack the selection cassette. In some embodiments, a method as described herein may further comprise a genotyping step after the first selection step (i.e., the positive selection step) after the second selection step, (i.e., the negative selection step), or after both selection steps. A genotyping step as described herein may comprise amplicon sequencing, and may be used to establish a pre- or post-selection efficiency parameter.

A cell population to be edited may be a cell sample from a patient or subject, for example a patient for whom a genomic edit may be beneficial or necessary to treat a given disease. A patient may be identified through a screening process in order to determine any impact on cell phenotype as a result of genomic editing. Following identification of a patient or subject in need, and prior to performing genomic editing in the patient in vivo, a preparatory procedure may be performed in vitro in a cell population, such that the cell population may already express a gene editing system as described herein prior to being introduced into the patient. Alternatively, depending on the individual needs of the patient, a gene editing system may be delivered to the patient in such a manner as to rely on the cellular machinery of the individual for expression of the components of the HDR cassette/vector. A cell population for introduction into a subject may be tested in an animal model, such as a murine, canine, porcine, simian, or the like (Platt et al., Cell 159:440-455, 2014). Any useful animal model may be used as appropriate with the invention and for the particular application.

In some embodiments, a method as described herein may further comprise determining changes in expression of one or more biomarkers in the final edited cell population and/or changes one or more cellular phenotypes of the final edited cell population. In some embodiments, the one or more changes in cellular phenotype may include changes in morphology, motility, cell death, cell-cell contact or a combination thereof. In some embodiments, one or more biomarkers as described herein are indicative of a presence or absence of a disease state. In other embodiments, one or more biomarkers may identify a cell type or cell lineage.

### Determining Efficiency of Editing

In accordance with the invention, determination of the efficiency of editing using the constructs, vectors, and methods as described herein is provided. For example, quantitative PCR (qPCR) may be performed using primers for a negative selection marker gene as described herein, such as the thymidine kinase. Internal control primers for sequences with known and stable copy number (e.g., RNase P) may be used to control for input cell number. Plasmid standard curves may be generated with the known copy number of the insert and control region using these primers. Such controls allow for absolute quantification of the fraction of cells containing the selection insert. When performed following positive selection, this fraction directly represents the editing efficiency (F1). When performed following negative selection, this fraction directly represents the rate of failed excision (F2). Overall editing efficiency may be calculated as (F1-F2).

### Combinatorial Editing of Genetic Variants

In some embodiments, the present invention may enable parallelized combinatorial editing of genetic variants by using up to six different positive selectable markers in tandem. Such an application may require the use of two different types of positive selection cassettes. For example, in one embodiment, one positive selection cassette may utilize an antibiotic resistance gene, and a second positive selection cassette may utilize a fluorescent tag.

Common selection agents applicable for all eukaryotes may include, but are not limited to, puromycin, blasticidin, geneticin (G-418), hygromycin B, among others. Selection agents such as zeocin may be used for mammalian/insect/yeast/plant applications. Applications relating only to plants may utilize, for example, bialaphos/BASTA, glyphosate, neomycin, or kanamycin, among others. Any appropriate selection marker for the particular application may be used as described above.

In some embodiments, one or both selection markers may be operably linked to a promoter for expression in the cell into which the gene is inserted. In other embodiments, both selection markers are operably linked to separate promoters for expression in a cell. In some embodiments, the elements of a HDR construct as described herein may be present on a single nucleic acid construct or a single vector. In other embodiments, such elements may be present on more than one construct or vector.

In some embodiments, a HDR construct as described herein may further comprise a screenable marker, such as green fluorescent protein (GFP), blue-white screening (lacZ) β-glucuronidase (GUS), luciferase (LUC), firefly luciferase (ff-LUC). Fluorescent markers as described herein may be used for fluorescence-activated cell sorting (FACS) in order to achieve isolation of positive cell pools, wherein the fluorescent marker comprises, for example, TagBFP (blue), Cerulean (cyan), Tag GFP2 (green), YPet (yellow), TagRFP (red), mKate2 (far red).

In other embodiments, thymidine kinase may be employed for negative selection in any construct in accordance with the invention, inducing cell death, if any other selection cassettes fail to excise a construct. This may enable creation of up to six parallel genomic edits in one cell pool. In other embodiments, drug selection and FACS-based isolation may be used, as well as a combination of these in order to provide additional possibilities. In such cases, cell survival would depend on incorporation of each expected resistance gene (and therefore would rely on successful editing), followed by scarless excision of the selection cassette. In some embodiments, a combinatorial editing system may be developed with the capability for both transfection and lentivirus delivery. Alternate embodiments may employ RNP, linearized plasmid, or non-linearized lentivirus delivery.

Combinatorial editing of three variants may be achieved using three HDR donor plasmids, each encoding a unique positive selection marker from the available sets of antibiotic resistance genes or fluorescent markers as described herein. In some embodiments, such an approach may be combined with the negative selection marker thymidine kinase. High efficiency combinatorial editing of all three variants in parallel in one cell pool may be achieved by positive selection with all three antibiotics and/or FACS sorting for cells in which successful homologous recombination of all three variants has occurred. Following positive selection, the excision-only PiggyBac transposase removes all selection cassettes without leaving any footprints. Cells containing all three accurate edits are negatively selected with FIAU which selects against cells still containing any of the selection cassettes.

When using the dual positive selection system for isolating homozygously-edited cell populations, a similar method may be used, but with primers for each of the positive selection markers rather than for the negative selection marker.

In some embodiments, combinatorial implementation may employ a combination of FACS-derived data (total cell count, cells per each combination of fluorescent markers per cell pool) and targeted genome sequencing. These data may be used to establish parameters for N-wise edit efficiencies per total individual edit efficiency and for excision efficiency. In some embodiments, a qPCR-based assay may be developed for relative efficiency quantification.

The invention is further described in the following examples, which do not limit the scope of the invention described in the claims.

### EXAMPLES

### Example 1—Vector Components

### Backbones:

The pFUGW-H1 empty vector was Addgene plasmid # 25870. (Fasano et al., Cell Stem Cell 1(1):87-99, 2007). The starting material pFUGW-H1 empty vector was an empty backbone 3rd generation lentiviral vector.

pBluescript II SK(+) Phagemid Kit (Agilent): f1 origin in (+) orientation, Sac->Kpn polylinker orientation, Contains: 20 μg pBluescript II SK(+) phagemid vector, Host Strain: XL1-Blue MRF′

PiggyBac transposon/Selection:
pPB-R1R2-NeoPheS→PiggyBac transposon elements
pENTR-PGKpuroΔtk→PGKpuroΔtk selection cassette

PGK promoter: constitutively active promoter, shown to be robust in human lymphocytes
Puro: Puromycin resistance gene for positive selection
ΔTK: Thymidine Kinase sensitization gene for negative selection

PiggyBac Transposase:
Excision only piggyBac Transposase expression vector
Cat. #: PB220PA-1 (Li et al., Proc Natl Acad Sci U S A. June 18; 110(25):E2279-87, 2013).

The transposase was used as intended, to deliver unaltered plasmid via Neon transfection. Subsequently, the transposase was removed by PCR, using known flanking sequences, and gibson cloned into pFUGW backbone as described above for lentiviral delivery of the excision-only transposase.

pCMV-hyPBase: The hyperactive PiggyBac transposase is not limited to excision.

### Example 2—Procedure for Double Selection Base Editing

Step 1: Construction of Targeting Vectors
Construct targeting vectors by cloning guide sequences into backbone containing Cas9 and sgRNA scaffold.
Timing: 3 d

Step 2: Preparation of sgRNA oligo insert
2.1) Resuspend the top and bottom strands of oligos for each sgRNA design (Step 1 above) to a final concentration of 100 μM.
2.2) Prepare the following mixture for phosphorylating and annealing the sgRNA oligos (top and bottom strands):
2.3) Phosphorylation and annealing of the oligos in a thermocycler by using the following parameters:
   37° C. for 45 min
   95° C. for 2.5 min
   Ramp down to 25° C. at 5° C./min
2.4) Dilute phosphorylated and annealed oligos 1:500 in room temperature ddH2O.

Step 3: Preparation of vector
3.1) Set up the following digestion reaction:
3.2) Incubate at 37° C. for 45 min.
3.3) Then add 1 of Fermentas FastAP and incubate for an additional 15 min.
3.4) Gel purify the vector.

Step 4: Ligation of sgRNA oligos into vector
4.1) Set up the following ligation reaction for each sgRNA:
   *Recommended: no-insert, vector-only negative control for ligation.
4.2) Incubate according to manufacturer's instructions. In general, 60-120 min at room temperature yields good results.

Step 5: Transformation (XL1-blue, Top10, DH5a)

Inspect the plates for colony growth. Typically, there are no colonies on the negative control plates (ligation of BbsI-digested pSpCas9(BB) alone without annealed sgRNA oligo insert), and there are tens to hundreds of colonies on the pSpCas9 (sgRNA) (sgRNA inserted into pSpCas9 (BB)) cloning plates.

Step 6: Check for correct insertion

From each plate, pick two or three colonies to check for the correct insertion of sgRNA. Use a sterile pipette tip to inoculate a single colony into a 3-mL culture of LB medium with 100 μg/mL ampicillin. Incubate the culture and shake at 37° C. overnight.

Step 7: Isolate plasmid DNA from cultures

QIAprep spin miniprep kit according to the manufacturer's instructions.

Step 8: Sequence validation of CRISPR plasmid

Verify the sequence of each colony by sequencing using the following primer: pLKO_U6_SEQ_fw: TTTGCTGTACTTTCTATAGTG (SEQ ID NO:2). Reference the sequencing results against the cloning vector sequence to check that the 20-nt guide sequence is inserted between the U6 promoter and the remainder of the sgRNA scaffold.

Step 9: Construction of HDR vectors
9.1) Digest inserts and backbone with BsaI and ligate resulting fragments.
9.1) Set up the following reaction referencing the tables for details for the appropriate parts:
   9.1.1) Mix appropriate volumes of your DNA segments together.
   9.1.2) Add water to a final volume of 14 μl.
   9.1.3) Add 2 μL of 10×T4 DNA ligase buffer and 2 μL 10×BSA. Mix by vortexing.
   9.1.4) Add 1 μL of BsaI and 1 μL of T4 DNA ligase. Mix by gently pipetting.
9.2) Run the reaction using the following program:

Step 10: Transformation. Transform into Stb13 or a comparable strain, or store reactions at 4° C. until ready to proceed to transformation.

Step 11: PCR

From each plate, perform direct colony PCR using two or three colonies, making sure to mark colonies and leave some bacteria for later inoculation) to check for the correct insertion of homology arms.

(SEQ ID NO: 3)
PB-F: CTGCTGCAACTTACCTCCGGGATG
(SEQ ID NO: 4)
PB-R: CCAATCCTCCCCCTTGCTGTCCTG
(SEQ ID NO: 5)
FUGW-F: CAGGGACAGCAGAGATCCAGT
(SEQ ID NO: 6)
FUGW-R: ACAATCAGCATTGGTAGCTGCTG

For pBluescript backbone, use PB primers with M13 F and R primers.

Step 12: Inoculation.

Inoculate a colony having a successful clone into a 3-mL culture of LB medium with 100 μg/mL carbenicillin. Incubate the culture and shake at 37° C. overnight.

Step 13: Isolation of plasmid

Isolate the plasmid DNA from cultures by using a QIAprep spin miniprep kit according to the manufacturer's instructions.

### Lentivirus Production:

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

### Prepare:

(1) HDR lentivirus using transfer vectors created in step 2 with IDLV packaging plasmid (psPAX2-D64V);
(2) Targeting vectors created in step 1 with packaging plasmid psPAX2;
(3) PiggyBac transposase virus using pre-constructed transfer vector with packaging plasmid psPAX2. Use envelope plasmid pVSVG for all.
All steps can be done at RT; dilute each plasmid to a concentration of 100 ng/μl prior to starting.

Step 14: Premix packaging (0.5 μg->5 μl) and envelope vector (0.5 μg->5 μl) by pipetting and by tapping the tube.

Step 15: Add transfer vector (vectors constructed in steps 1 & 2 above; PiggyBac transposase vector) (1.0 μg->10 μl).

Step 16: Premix 12 μl FuGene with 100 μl OPTIMEM and mix by vortexing.

Step 17: Add FuGene mixture to plasmid mixture and vortex

Step 18: Incubate mixture for 15-25 min. In the meantime, prepare HEK293T cells (Steps 19-24).

Step 19: Wash the cells 1× with PBS (do not pipette up and down), and remove PBS with a vacuum pump.

Step 20: Add 5 ml Trypsin to a 60-mm plate and incubate at 37° C. for 5 min.

Step 21: Stop with 10 ml DMEM, pipette the suspension to a 50-ml tube and mix by pipetting up and down.

Step 22: Centrifuge cells at 500×g for 5 min.

Step 23: Resuspend cells and calculate cells, taking care that the cells are alive by ensuring that there is no inclusion of Trypan blue.

Step 24: Dilute 3.8×10⁶ cells in 1 mL, to obtain a final concentration of 1.8×10⁶ cells in 500 μl.

Step 25: Prepare 1 mL of pre-warmed medium in each well of a 6-well plate.

Step 26: Mix transfection mixture from step 5 with prepared cells.

Step 27: Add 600 μl of mixture to each well containing already pre-warmed medium.

Step 28: Change medium on the second day (˜18 h), using a medium compatible with cells that will be infected.

Step 29: Incubate for 48 h total (each well can produce around 2 ml virus).

Step 30: Collect supernatant with a 0.45 μm syringe filter. The virus is ready to use for transduction after being filtered. Transduction

Step 31: Transduction Day 1 (AM): Spin down 1×10⁶ cells per condition cells in 50 ml polypropylene falcon tubes. Allow for 2×2 control wells (+polybrene-virus, −polybrene-virus).

Step 32: Prepare a mixture of pre-warmed PBMC basal stimulation medium containing 8 ug/mL polybrene (final concentration in plates will be 5.2 μg/mL).

Step 33: Resuspend in 650 μL prepared medium+polybrene per condition and add 650 μL cells to each well of 24-well plates.

Step 34. Add 250 μL of respective HDR lentivirus supernatant and 100 μL of respective Cas9-sgRNA lentivirus supernatant to each well.

Step 35: Incubate for 8 h in standard incubation conditions.

Step 36: Transduction Day 1 (PM): Following transduction, spin down cells, wash once with PBS, and resuspended in fresh basal stimulation medium.

(+) Selection: Puromycin positive selection for successfully edited cells.

Step 37: At day 4 (PM) after transduction, replace the medium with medium containing previously optimized selection concentration of puromycin (0.6 ug/mL).

Step 38: Replace the medium with basal medium containing 0.6 ug/mL puromycin on day 6 (PM).

Step 39: From day 8 (PM) until excision, resistant colonies should be maintained with medium containing 0.2 ug/mL puromycin, replaced every other day.

Step 40. When cells have expanded a bit and look somewhat recovered (˜day 11): Split, re-plate in standard medium (no puro) for excision, and collect fraction of cells (for genotyping).

### Genotyping

Step 41: Add ˜300-500 k cells to a 1.5 ml microcentrifuge tube and spin down at 500 g for 5 minutes.

Step 42: Remove medium, wash gently with PBS.

Step 43: Aspirate as much of the supernatant as possible without disturbing the cell pellets.

Step 44: Lyse cells by adding 50 μL of QuickExtract DNA Extraction Solution.

Step 45. Transfer cell lysate to appropriate PCR tubes or plate.

Step 46: Vortex (2×20 sec) and heat in a heating block (or thermal cycler) at 65° C. for 15 min, remove and vortex again (2×20 sec) and then heat in a heating block (or thermal cycler) at 95° C. for 15 min.

Step 47: Add 100 μL of Nuclease-Free Water to dilute the genomic DNA.

Step 48: Vortex and spin down.

Step 49: For each condition, set up a PCR reaction following the “Genotyping” protocol, as follows:

** Every time: run “standard” (e.g., 1×104 molecules of HDR plasmid) to control for variance between runs.

** Extracted gDNA from 300-500k cells using QuickExtract should yield ˜1.5 ug total=>˜30 ng/uL.

Set up the following reaction in duplicate:

Run with the following program:

Step 50: Following analysis, proceed with successfully edited cell pools.

### Excision

Step 51: Transposon Removal. Infection with lentivirus produced in “Lentivirus Production” section above, following previously detailed “Transduction” protocol.

(−) Selection: FIAU negative selection for successful excision of selection cassette.

Step 52: On day 4 (PM) after transduction, start FIAU selection. Change to medium containing previously optimized 1 ug/mL of FIAU. As cells grow, a daily medium change may be required depending on the number of surviving cells.

Step 53. Collect one fraction of cells for genotyping, one to freeze down, and re-plate remainder.

### Genotyping

Step 54: Repeat steps from previous “Genotyping” section with the cells that survive negative selection.

### Example 3—Determination of Efficiency of Double-Selection Base Editing

To quantify efficiency following double-selection base editing, gDNA is extracted from a fraction of cells per condition and qPCR is performed with primers for the thymidine kinase (negative selection) insert. Internal control primers for sequences with known and stable copy number (e.g., RNase P) are used to control for input cell number. Plasmid standard curves are first generated with known copy number of the insert and control region using these primers. This is performed only once, and for each subsequent round/condition, a plasmid sample of known copy number is used to control for variance between runs. These controls allow for absolute quantification of the fraction of cells containing the selection insert. When performed following positive selection, this fraction directly represents the editing efficiency (F1). When performed following negative selection, this fraction directly represents the rate of failed excision (F2). Overall editing efficiency therefore can be calculated as (F1-F2).

When using the dual positive selection system for isolating homozygously-edited cell populations, a similar method is used, but with primers for each of the positive selection markers, rather than for the negative selection marker.

For the combinatorial implementation, a combination of FACS-derived data (total cell count, cells per each combination of fluorescent markers per cell pool) and targeted genome sequencing is used. These data are used to establish parameters for N-wise edit efficiencies per total individual edit efficiency and for excision efficiency. This enables development of a qPCR-based assay for relative efficiency quantification, which will be suitable for use in future studies.

### Example 4—Combinatorial Editing

Parallelized combinatorial editing of genetic variants can be performed by using up to six different positive selectable markers in tandem. Two different types of positive selection cassettes are created, one of which utilizes antibiotic resistance. Common selection agents may include the following:

All eukaryotes: Puromycin, Blasticidin, Geneticin (G-418), Hygromycin B.

Mammalian/Insect/Yeast/Plants: Zeocin.

Plants: Bialaphos/BASTA, Glyphosate, neomycin, kanamycin.

In addition, selection agents may use fluorescent tags, such as Blue-TagBFP, Cyan-Cerulean, Green-Tag GFP2, Yellow-YPet, Red-TagRFP, Far Red-mKate2. As already optimized in the high efficiency CRISPR/Cas9 variant editing approach, thymidine kinase can be used for negative selection in all constructs, inducing cell death if any of the selection cassettes fail to excise. This allows creation of up to six parallel edits in one cell pool. In addition, if only drug selection or FACS-based isolation are used, more possibilities are available by combining the two approaches. In such a case, cell survival depends on incorporation of each expected resistance gene (and therefore edit), followed by scarless excision of the selection cassette. As with the approach described above at the single variant level, the combinatorial editing system is developed with the capability for both transfection and lentivirus delivery.

Combinatorial editing of three variants is achieved by three HDR donor plasmids, each encoding a unique positive selection marker from available antibiotic resistance genes or fluorescent markers combined with the negative selection marker thymidine kinase. High efficiency combinatorial editing of all three variants in parallel in one cell pool is achieved by positive selection with all three antibiotics and/or FACS sorting for cells in which successful homologous recombination of all three variants has occurred. Following positive selection, the excision-only PiggyBac transposase removes all selection cassettes without leaving any footprints. Cells containing all three accurate edits are negatively selected with FIAU, which selects against cells still containing any of the selection cassettes.

### Example 5—CRISPR-SAVE Basic Protocol

This protocol assumes use of lentiviral delivery and lentivirus reagents. If transfection is preferred, use transfection-ready HDR backbone, disregard lentivirus production step (3.1), and use transfection protocols appropriate for your cell type.
1. HOMOLOGY ARM DESIGN AND VECTOR ASSEMBLY
   1.1. Design homology arm primers manually or using the design GUI, following the respective instructions:
   1.1.1. Use construct designer GUI to design homology arm primers for all intended edits following associated protocol.
   1.1.2. Design manually according to the following instructions:
      1.1.2.1 Retrieve genomic sequence from UCSC genome browser using the rs number of the SNP you want to edit. Take 2 kb upstream and downstream of the variant.
         1.1.2.1.1. Get FASTA sequence: View->DNA->2000 bases up and down->highlight common SNPs->submit; put sequences into text file.
      1.1.2.2. Search sequence +/−300 bp of desired edit for any instances of ‘TTAA’. (This TTAA site will be used for piggyBac transposon insertion.)
      1.1.2.3. If there are multiple TTAA' sites within this region, prioritize by (1) distance to available PAM site (ideally close enough for guide sequence to overlap ‘TTAA’, generally no more than 100 bp away) and (2) distance to intended edit.
      1.1.2.4. Select sequence +700 bp (HA-R) and −700 bp (HA-L) of top-ranked ‘TTAA’ site.
      1.1.2.5. Design genomic primers using Primer3 to amplify each homology arm.
      1.1.2.6. Add the following overhangs to the primers:

HA_L:
Forward primer:
(SEQ ID NO: 7)
5′ GCTAGCTAGGTCTCCCAGA (annealing sequence) 3′
Reverse primer:
(SEQ ID NO: 8)
5′ CGTACGTAGGTCTCCAAGC[TT] (annealing sequence) 3′
HA_R:
Forward primer:
(SEQ ID NO: 9)
5′ GCTAGCTAGGTCTCCAGGT[TT] (annealing sequence) 3′
Reverse primer:
(SEQ ID NO: 10)
5′ CGTACGTAGGTCTCCGTTG (annealing sequence) 3′

1.2. Isolate genomic DNA from cells you will be editing using the QuickExtract reagent following standard recommended protocol.
1.2.1. Use 5 or less of the extracted DNA for each PCR amplification
1.3. Prepare homology arms.
1.3.1. Re-suspend primers and make aliquots with concentration of 25 uM.
1.3.2. Set up the following reaction for each homology arm using Q5 High-Fidelity 2X Master Mix following manufacturer's protocol and run PCRs using the recommended conditions:

TABLE 9

| Reagent | Volume |
|---|---|
| gDNA (QuickExtract) | 2 uL |
| Primer F (25 uM) | 1 uL |
| Primer R (25 uM) | 1 uL |
| 2X Q5 mastermix | 22.5 uL |
| Nuc free H20 | 23.5 uL |
| Total | 50 uL |

TABLE 10

| Temperature | Time | Cycles |
|---|---|---|
| 98° C. | 30 sec | 1x |
| 98° C. | 10 sec | 1x |
| Gradient* | 20 sec | 30-35x |
| 72° C. | 30 sec | 1x |
| 72° C. | 2 min | 1x |
| 4° C. | hold | |

(*based on Tm range of HA primers—use columns within 0.3-0.4 deg of the target temperature)

1.3.3. Verify products are specific and of intended size by gel electrophoresis of approximately 2 ul of each PCR product.
1.3.4. Gel extract and purify homology arms from remaining product.
1.3.5. Clone into pMini 2.0 following standard protocol for NEB PCR Cloning Kit (using vector:insert ratio of approximately 3:1)

TABLE 11

| Reagent | Volume |
|---|---|
| Linearized pMiniT 2.0 | 1 μl |
| Vector (25 ng/μl) 2.6 kb | (25 ng) |
| Insert* | 1-4 μl* |
| H20 | to 5 μl |
| Cloning Mix 1 | 4 μl |
| Cloning Mix 2 | 1 μl |
| Total | 10 μl |

*Will depend on conc.

1.3.6. Transform plasmids into chemically competent E.coli, plate transformed cells onto LB plates containing ampicillin, pick colonies and inoculate liquid cultures, then isolate and purify plasmid DNA.
1.3.7. Sequence plasmids to verify insertion of homology arms and to determine which arms will require mutagenesis to create the specific allele you want after editing (eg. your template has a T, but you want to edit a T->C in your target cell, than you mutate T->C in your plasmid).
   1.3.7.1. If WT arms contain desired variant, move on to cloning into CRISPR-SAVE backbone.
   1.3.7.1. Otherwise, perform site-directed mutagenesis to convert variant to desired allele.
      1.3.7.2.1. Select mutagenesis kit based on the number of mutations needed per arm.
      1.3.7.2.2. Use NEBaseChanger or QuikChange Primer Design Program to design mutagenesis primers.
      1.3.7.2.3. Design variant mutation primers: use genomic sequence around the variant, select variant and mutation.
      1.3.7.2.4. Use the QuikChange Lightning or NEB Q5 Site-Directed Mutagenesis kit to mutate, following manufacturer's instructions.
      1.3.7.2.5. Transform mutated plasmids into chemically competent e.coli, plate transformed cells onto LB plates containing ampicillin, pick colonies and inoculate liquid cultures, then isolate plasmid DNA and send for sequencing to verify mutagenesis.
1.4 Clone into CRISPR-SAVE backbone.
1.4.1. Set up golden-gate cloning reaction and run reaction in a thermal cycler using the following conditions:

TABLE 12

| Reagent | Amount | Volume |
|---|---|---|
| pFUGW-PuroTk- | 100 ng | — |
| EGFP | | |
| pMini.HA | 2:1 Molar ratio | — |
| | insert to plasmid, | |
| | (72 ng of each) | |
| NEB Golden Gate | | 2 uL |
| Buffer 10X | | |
| NEB Golden Gate | | 1 uL |
| Assembly Mix | | |
| Nuclease-free H20 | | to 20 uL |
| Total | | 20 uL |

TABLE 13

| Temperature | Time | Cycles |
|---|---|---|
| 37° C. | 15 min | 1x |
| 37° C. | 2 min | 50x |
| 16° C. | 5 min | |
| 37° C. | 15 min | 1x |
| 50° C. | 5 min | 1x |
| 80° C. | 5 min | 1x |
| 65° C. | 20 min | 1x |
| 4° C. | hold | hold |

1.4.2. Transform mutated plasmids into chemically competent e.coli (such as NEB Stable Competent E. coli) and plate transformed cells onto LB plates containing ampicillin.
1.4.3. Perform colony PCRs (about 10 per each construct should be sufficient) to check for successful incorporation of left and right homology arms and run colony PCRs using the following conditions:

TABLE 14

| Reagent | 1X |
|---|---|
| Template (colony)* | — |
| FUGW-HA-R-For (25 uM) | 1 uL |
| FUGW-HA-R-Rev (25 uM) | 1 uL |
| 2X One Taq Hot start | 12.5 uL |
| Nuclease free H2O | 10.5 uL |
| Total | 25 uL |

*Mark selected colonies, collect half of each selected colony and add directly to the 10.5 uL of Nuclease free H2O

TABLE 15

| Temperature | Time | Cycles |
|---|---|---|
| 94° C. | 2 minutes | 1x |
| 94° C. | 15 seconds | |
| 52° C. | 15 seconds | 30x |
| 68° C. | 60 seconds | |
| 68° C. | 5 minutes | 1x |
| 4-10° C. | hold | Hold |

2. GUIDE DESIGN AND VECTOR ASSEMBLY
   2.1. Use designer GUI to design guide RNA sequences for your set of variants.
   2.2. Prepare sgRNA oligos:
   2.2.1. Resuspend the top and bottom strands of oligos for each sgRNA design (Step 1) to a final concentration of 100 μM.
   2.2.2. Prepare the following mixture for phosphorylating and annealing the sgRNA oligos (top and bottom strands):

TABLE 16

| Prepare the following mixture for | |
|---|---|
| phosphorylating and annealing the sgRNA | Amount |
| oligos (top and bottom strands):Component | (μl) |
| sgRNA top (100 μM) | 1 |
| sgRNA bottom (100 μM) | 1 |
| T4 ligation buffer, 10× | 1 |
| (not T4 PNK buffer) | |
| T4 PNK | 0.5 |
| ddH2O | 6.5 |
| Total | 10 |

2.2.3. Phosphorylate and anneal the oligos in a thermocycler by using the following parameters:

TABLE 17

| Temperature | Time |
|---|---|
| 37° C. | 45 min |
| 95° C. | 2.5 min |
| 25° C. | Ramp down |
| | at 5° C. min − 1 |

2.2.4. Dilute phosphorylated and annealed oligos 1:500 in room temperature ddH2O.
2.3. Prepare the vector.
2.3.1. Set up the following digestion reaction and incubate at 37° C. for 45 min.

TABLE 18

| Component | Amount (μl) |
|---|---|
| 2 μg vector | x |
| FastDigest buffer | 2 |
| FastDigest Esp3I (BsmBI) | 1 |
| DTT (10 mM) | 2 |
| H20 | x |
| Total | 20 |

2.3.2. Then add 1 ul of Fermentas FastAP and incubate for an additional 15 min.
2.3.3. Gel purify the vector.
2.4. Ligate sgRNA oligos into vector.
2.4.1. Set up the following ligation reaction for each sgRNA:

TABLE 19

| Components: | Amount (μl): |
|---|---|
| Vector (~60-100 ng) | x |
| Oligo duplex | 2 |
| T4 DNA Ligase buffer | 1 |
| T4 DNA Ligase | 1 |
| H2O | 6 |
| Total | 10 |

2.4.2. Incubate according to manufacturer's instructions. In general 60-120 min at RT yields good results.
2.5. Transform mutated plasmids into chemically competent E.coli (such as NEB Stable Competent E. coli), plate transformed cells onto LB plates containing ampicillin, pick colonies and inoculate liquid cultures, then isolate plasmid DNA and send for sequencing to verify correct insertion of homology arms.
   NOTE: Before starting this phase of experiments, it is important to optimize lentivirus transduction conditions and positive/negative selection conditions for your cell type.
3. EDIT TARGET CELL POPULATION
   3.1. Lentivirus Production
Prepare lentivirus for HDR template construct (e.g. pFUGW-PuroTk-EGFP) and sgRNA construct (pL-CRISPR.SFFV.GFP, addgene plasmid #57827):
   Day 0 : Seed 293T packaging cells in antibiotic free media −3.3×106K cells/ 6 well plate (275K cells/mL in a total vol of 12 mL)
   Day 1 (pm): Transfect lenti constructs in to 293T cells
      1. (˜1 hour before starting) Warm OptiMEM and Fugene to room temperature.
      2. Briefly vortex Fugene.
      3. Add dVPR, VSVG, and transfer plasmid volumes according to table above directly to a tube containing 50 uL of OptiMEM
      4. Combine OptiMEM and Fugene, mix well by flicking. Incubate for 2-5 min RT Add OptiMEM FIRST, then add Fugene directly into OptiMEM (not against the side of the tube).
      5. Add OptiMEM/Fugene to DNA mix. Flick to mix. DO NOT VORTEX.
      6. Incubate 15 min at RT, max time of 20 min
      7. Add slowly and dropwise to cells.
      8. Rock plate back-and-forth and side-to-side gently to mix. Do not Swirl.
      9. Return plate to incubator.
   Day 2 (am): 18 hours post transfection. Remove media, replace with fresh media containing antibiotics.
   Day 3 (am): Harvest virus. Filter with 0.45 um syringe filter. Virus is now ready to use for transducing cells.
   3.2 Lentivirus infection of target cells with HA and guide vectors
      This will be specific to cell type of interest—transduce target cells following best practices for your cell type.
   3.3. Positive selection
   3.3.1. Drug selection
      3.3.1.1. Using concentrations and timing optimized for cell type of interest, perform selection with puromycin and/or other antibiotic (if using multiple different selection cassettes) to kill cells which have not incorporated the selection cassette(s).
   3.3.2. FACS (for initial sort and/or to assess efficiency)
      3.3.2.1. If using multiple different selection cassettes for bi-allelic editing, perform FACS and gate for cells containing both fluorophores. Otherwise gate for single color.
   3.4. (Optional) Assess edited pool for rate of correct on-target edit
      3.4.1.1. Set up the following PCRs to amplify target genomic regions using primers from the design GUI:
         1. HA-L-external+selection-rev
         2. HA-R-external+selection-fwd
         3. HA-L-external+HA-R-external
      3.4.1.2. Prepare and send PCR products for sequencing to assess (1) indel rate, (2) frequency of desired variant, and (3) proportion of cells containing the selection cassette.
   3.5. Lentivirus infection of target cells with transposase vector
      This will be specific to cell type of interest—transduce target cells following best practice for your cell type.
   3.6. Negative selection
   3.6.1. Drug selection
      3.6.1.1. Using concentrations and timing optimized for cell type of interest, perform selection with FIAU to kill cells which still contain the selection cassette
   3.6.2. FACS (for initial sort and/or to assess efficiency)
      3.6.2.1. Perform FACS, gating for no fluorescence to recover double excised cells.
   3.7. Assess final edited pool for off-target edits and rate of correct on-target edit
   3.7.1. Off-target
      3.7.1.1. Use preferred method to asses off-target mutation rate (e.g. computationally predict top n likely off-target sites for each guide and sequence those sites).
   3.7.2. On-target
      3.7.2.1. Set up the following PCRs to amplify target genomic regions using primers from the design GUI:
      1. HA-L-external+selection-rev
      2. HA-R-external+selection-fwd
      3. HA-L-external+HA-R-external
      3.7.2.2. Prepare and send PCR products for sequencing to assess (1) indel rate, (2) frequency of desired variant, and (3) proportion of cells still containing the selection cassette.
4. DOWNSTREAM FUNCTIONAL EXPERIMENTS, PHENOTYPING
   4.1.1. E.g. VCR of predicted target genes, scRNA-Seq, high-content imaging, functional assays.

### Reagents

Plasmids:
   a. HDR backbones:

(SEQ ID NO: 11)
i. pFUGW-SAVE-PuroTk-EGFP*

1. Or with any other drug resistance genes in place of puromycin resistance gene
2. Or with any other fluorescent markers in place of EGFP

(SEQ ID NO: 12)
ii. pMini-SAVE-PuroTk-EGFP**

1. Or with any other drug resistance genes in place of puromycin resistance gene
2. Or with any other fluorescent markers in place of EGFP

(SEQ ID NO: 13)
iii. pFUGW-CMV-hyPBase-ExcOnly-IRES-GFP***

* Addgene (standard Broad MTA with Addgene)
pFUGW-H1 empty vector was a gift from Sally Temple (Addgene plasmid # 25870) [shRNA knockdown of Bmi-1 reveals a critical role for p21-Rb pathway in NSC self-renewal during development. Fasano Calif., Dimos J T, Ivanova N B, Lowry N, Lemischka I R, Temple S. Cell Stem Cell. 2007 Jun. 7. 1(1):87-99.10.1016/j stem.2007.04.001 PubMed 18371338]

### ** NEB PCR Cloning Kit, #E1202S

*** The construct expressing the hyperactive PB transposase (pCMV-hyPBase) has been described previously and was provided by Allan Bradley (Wellcome Trust Sanger Institute, Cambridge, UK) and Nancy Craig (The Johns Hopkins University School of Medicine, Baltimore, Md., USA)—MTA in place.
[A hyperactive piggyBac transposase for mammalian applications. Yusa K, Zhou L, Li M A, Bradley A, Craig N L, Proc Natl Acad Sci U S A. 2011 Jan. 25; 108(4):1531-6.]
Commercially Available:
   1. Plasmids
      a. pL-CRISPR.SFFV.GFP (Cas9+sgRNA backbone) (addgene plasmid #57827)
      b. pSpCas9(BB)-2A-GFP (PX458, addgene plasmid # 48138)
      c. Envelope plasmid (pCMV-VSV-G; addgene plasmid # 8454)
      d. Packaging plasmid (integrative lentivirus) (psPAX2; addgene plasmid #12260)
      e. Packaging plasmid (integrative deficient lentivirus (IDLV)) (psPAX2-D64V; addgene plasmid #63586)
   2. Reagents
      a. Q5 High-Fidelity 2X Master Mix (NEB)
      b. NEB PCR Cloning Kit
      c. NEB Q5 Site-Directed Mutagenesis kit
      d. NEB Golden Gate Assembly kit
      e. 2X OneTaq Hot Start
      f. Qiagen plasmid plus maxi kit (Qiagen)
      g. QIAquick PCR purification kit (Qiagen)
      h. QIAquick gel extraction kit (Qiagen)
      i. QIAprep spin miniprep kit (Qiagen)
      j. T4 PNK
      k. DTT, 10 mM
      l. Fermentas FastAP
      m. T4 DNA Ligase+buffer (NEB)
      n. FIAU (Moravek, cat. no. M251)
      o. Puromycin Dihydrochloride (Thermo Scientific, cat. No. A1113803)
      p. QuickExtract DNA Extraction Solution (Epicentre)
      q. BsaI (Thermo Scientific or NEB)
      r. CutSmart buffer (NEB)
      s. FastDigest buffer (Thermo Scientific)
      t. FastDigest Esp3I (BsmBI) (Thermo Scientific)
      u. High Efficiency Transformation for NEB® Stable Competent E. coli (C3040H) and associated outgrowth requirements (LB, plates, Ampicillin, etc.)
   3. Cell culture: specific to cell type of interest

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A homology directed repair (HDR) construct for variant screening in cells comprising:
a left and right homology arm, with either the left or right homology arm encoding a genomic edit to be incorporated at a target locus; and
an excisable double selection cassette located within the left and right homology arms, the excisable double selection cassette comprising;
a first selection marker; and
a second selection marker; and
wherein the first selection marker and the second selection marker are located between a first and second excision site.

2. The HDR construct of claim 1, wherein the first and second selection markers are positive selection markers, or negative selection markers.

3. The HDR construct of claim 1 or 2, wherein the first or second selection marker, or both, is a zeocin resistance gene, a blasticidin resistance gene, a geneticin (G-418) resistance gene, or a hygromycin B resistance gene.

4. The HDR construct of claim 1, further comprising a fluorescent marker for FACS isolation of positive cell pools, wherein the fluorescent marker comprises Blue-TagBFP, Cyan-Cerulean, Green-Tag GFP2, Yellow-YPet, Red-TagRFP, Far Red-mKate2.

5. The HDR construct of any of claims 1 to 4, wherein the left and right homology arms are each from about 700 bp to about 1000 bp.

6. The HDR construct of claim 1, wherein the first selection marker is a drug resistance gene.

7. The HDR construct of claim 6, wherein the drug resistance gene is a puromycin resistance gene.

8. The HDR construct of claim 1, wherein the second selection maker is a drug sensitivity gene.

9. The HDR construct of claim 8, wherein the drug sensitivity gene is a thymidine kinase.

10. The HDR construct of any of claims 1 to 9, wherein the first and second excision sites are transposase recognition sites.

11. A homology directed repair (HDR) vector comprising the construct of any one of claims 1 to 10.

12. The vector of claim 11, wherein the backbone of the vector enables uniform, one-step assembly for incorporating homology arms.

13. The HDR vector of claim 11, wherein the vector is a transfection delivery vector.

14. The HDR vector of claim 11, wherein the vector is a viral delivery vector.

15. The HDR vector of claim 14, wherein the viral delivery vector is a lentivirus vector

16. A variant screening system for screening cells comprising:
a gene editing system;
a HDR vector of any one of claims 11 to 15; and
an excision protein or a polynucleotide encoding an excision protein, wherein the excision protein removes the excisable double selection cassette.

17. The system of claim 16, wherein the gene editing system comprises a CRISPR system comprising a CRISPR effector protein and/or a polynucleotide encoding the CRISPR effector protein, and a guide RNA (gRNA) comprising a guide sequence and/or a polynucleotide encoding the gRNA, wherein the gRNA is capable of forming a complex with the CRISPR effector protein and binding a target sequence adjacent to a variant locus to be edited.

18. The system of claim 16, comprising two or more delivery vectors, each delivery vector comprising a guide RNA targeted to a different variant locus.

19. The system of any of claims 16 to 18, comprising two or more HDR vectors wherein each HDR vector encodes a different nucleotide edit at each variant locus.

20. The system of any of claims 16 to 19, wherein the excision protein is a transposase.

21. The system of claim 20, wherein the transposase is an excision transposase.

22. The system of claim 20, wherein the transposase is a hyperactive transposase.

23. The system of claim 20, wherein the transposase comprises a mutation that alters its function.

24. The system of claim 20, wherein the transposase comprises a PiggyBac transposase.

25. A method for screening variant loci in cells comprising;
delivering one or more HDR constructs of any one of claims 1 to 10 and/or one or more HDR delivery vectors of anyone of claims 11 to 15 to;
(i) a population of cells expressing a gene editing system configured to cut cellular DNA at one or more target loci; or
(ii) a population of cells to which a gene editing system configured to cut cellular DNA at one or more target loci is co-delivered with the HDR construct or the HDR delivery vector;
selecting edited cells that incorporate the excisable double selection cassette of the HDR construct based on the first selection marker;
selecting a final cell population based on the second selection marker; and
delivering an excision protein, or a polynucleotide encoding the excision protein, to the edited cells, wherein the excision protein removes the excisable double selection cassette, to arrive at a final edited cell population.

26. The method of claim 25, wherein the gene editing system comprises a CRISPR system.

27. The method of claim 25, further comprising a genotyping step after the first selecting step, the second selecting step, or both.

28. The method of claim 27, wherein the genotyping step can be used to establish a pre- or post-selection efficiency parameter.

29. The method of claim 27, wherein the genotyping step comprises amplicon sequencing.

30. The method of any of claims 25 to 29, further comprise determining changes in expression of one or more biomarkers in the final edited cell population and/or changes one or more cellular phenotypes of the final edited cell population.

31. The method of claim 30, wherein the one or more changes in cellular phenotype include changes in morphology, motility, cell death, cell-cell contact or a combination thereof.

32. The method of claim 30, wherein the one or more biomarkers are indicative of a presence or absence of a disease state or identify a cell type or cell lineage.
